(19) United States
(12) Patent Application Publication
(10) Pub. No.: US 2004/0185524 A1
(43) Pub. Date: Sep. 23, 2004

(54) Biological samples and method for increasing survival of biological samples

(76) Inventors: John H. Crowe, Davis, CA (US); Fern Tablin, Davis, CA (US); Kamran Jamil, Woodland Hills, CA (US); Ann E. Oliver, Sacramento, CA (US)

Correspondence Address: CARPENTER & KULAS, LLP, 1900 EMBARCADERO ROAD, SUITE 109, PALO ALTO, CA, 94303, US

(21) Appl. No.: 10/721,678

(22) Filed: Nov. 25, 2003

Related U.S. Application Data
(63) Continuation-in-part of application No. 10/052,162, filed on Jan. 16, 2002, now Pat. No. 6,770,478.
Application No. 10/052,162 is a continuation-in-part of application No. 09/927,760, filed on Aug. 9, 2001.
Application No. 10/052,162 is a continuation-in-part of application No. 09/828,627, filed on Apr. 5, 2001, now Pat. No. 6,723,497.
Application No. 10/052,162 is a continuation-in-part of application No. 09/501,773, filed on Feb. 10, 2000, now abandoned.

(51) Int. Cl. G01N 1/30; G01N 33/48

(52) U.S. Cl. 435/40.5; 435/317.1

(57) ABSTRACT

A method for loading a biological sample comprising loading a biological sample with a solute and dimethylsulfoxide (DMSO) by fluid phase endocytosis to produce an internally loaded biological sample. A dehydrated composition is provided that includes dried biological samples containing dimethylsulfoxide and a solute. A method for increasing the survival of biological samples comprising providing biological samples, loading the biological samples with a carbohydrate and dimethylsulfoxide to produce loaded biological samples, and drying (e.g., air drying or vacuum drying) the loaded biological samples while maintaining a residual water content in the biological samples of at least about 0.01 gram water per gram of dry weight of biological samples to increase survival of the biological samples upon rehydrating.

### STATEMENT REGARDING FEDERAL SPONSORED RESEARCH AND DEVELOPMENT

Embodiments of this invention were made with Government support under Grant No. N66001-02-C-8055, awarded by the Department of Defense Advanced Research Projects Agency (DARPA). Further embodiments of this invention were made with Government support under Grant Nos. HL57810 and HL61204, awarded by the National Institutes of Health. The Government has certain rights to embodiments of this invention.

### FIELD OF THE INVENTION

Embodiments of the present invention generally broadly relate to biological samples, such as mammalian cells, platelets, and the like. More specifically, embodiments of the present invention generally provide for the preservation and survival of biological samples.

Embodiments of the present invention also generally broadly relate to the therapeutic uses of biological samples; more particularly to manipulations or modifications of biological samples, such as loading biological samples with solutes (e.g., carbohydrates, such as trehalose) and preparing dried compositions that can be re-hydrated at the time of application. When biological samples for various embodiments of the present invention are re-hydrated, they are immediately restored to viability.

The compositions and methods for embodiments of the present invention are useful in many applications, such as in medicine, pharmaceuticals, biotechnology, and agriculture, and including transfusion therapy, as hemostasis aids and for drug delivery.

### BACKGROUND OF THE INVENTION

A biological sample includes cells and blood platelets. A cell is typically broadly regarded in the art as a small, typically microscopic, mass of protoplasm bounded externally by a semi-permeable membrane, usually including one or more nuclei and various other organelles with their products. A cell is capable either alone or interacting with other cells of performing all the fundamental function(s) of life, and forming the smallest structural unit of living matter capable of functioning independently.

Cells may be transported and transplanted; however, this requires preservation which includes drying (e.g., vacuum drying, air drying, etc.), freezing and subsequent reconstitution (e.g., thawing, re-hydration, etc.) after transportation. Unfortunately, a very low percentage of cells retain their functionality after undergoing freezing and thawing. While some protectants, such as the cryoprotectant such as dimethylsulfoxide, tend to lessen the damage to cells, they still do not prevent some loss of cell functionality.

Blood platelets are typically generally oval to spherical in shape and have a diameter of 2-4 μm. Today platelet rich plasma concentrates are stored in blood bags at 22° C.; however, the shelf life under these conditions is limited to five days. The rapid loss of platelet function during storage and risk of bacterial contamination complicates distribution and availability of platelet concentrates. Platelets tend to become activated at low temperatures. When activated they are substantially useless for an application such as transfusion therapy. Therefore, the development of preservation methods that will increase platelet lifespan is desirable.

Trehalose has been found to be suitable in the preservation of cells and platelets. Trehalose is a disaccharide found at high concentrations in a wide variety of organisms that are capable of surviving almost complete dehydration. Trehalose has been shown to stabilize membranes, proteins, and certain cells and platelets during drying (e.g., freeze-drying) in vitro.

Spargo et al., U.S. Pat. No. 5,736,313, issued Apr. 7, 1998, have described a method in which platelets are loaded overnight with an agent, preferably glucose, and subsequently lyophilized. The platelets are preincubated in a buffer and then are loaded with carbohydrate, preferably glucose, having a concentration in the range of about 100 mM to about 1.5 M. The incubation is taught to be conducted at about 10° C. to about 37° C., most preferably about 25° C.

U.S. Pat. No. 5,827,741, Beattie et al., issued Oct. 27, 1998, discloses cryoprotectants for human cells and platelets, such as dimethylsulfoxide and trehalose. The cells or platelets may be suspended, for example, in a solution containing a cryoprotectant at a temperature of about 22° C. and then cooled to below 15° C. This incorporates some cryoprotectant into the cells or platelets, but not enough to prevent hemolysis of a large percentage of the cells or platlets.

Accordingly, a need exists for the effective and efficient preservation of biological samples, such as platelets and cells, and the like., More specifically, and accordingly further, a need also exists for the effective and efficient preservation of platelets and cells (e.g., erythrocytic cells, eukaryotic cells, or any other cells, and the like), such that the preserved platelets and cells respectively maintain their biological properties and may readily become viable after storage.

### SUMMARY OF EMBODIMENTS OF THE INVENTION

In one aspect of the present invention, a dehydrated composition is provided comprising dried biological sample(s) (e.g., freeze-dried platelets and cells) that are effectively loaded with a solute (e.g., trehalose) to preserve biological properties during drying, freezing and rehydration. Biological samples comprising platelets are rehydratable so as to have a normal response to at least one agonist, such as thrombin. For example, substantially all freeze-dried platelets for various embodiments of the invention when rehydrated and mixed with thrombin (1 U/ml) form a clot within three minutes at 37° C. The dehydrated biological sample(s) may include one or more other agents, such as antibiotics, antifungals, growth factors, or the like, depending upon the desired therapeutic application.

Embodiments of the present invention provide a process for loading a biological sample comprising loading a biological sample with a solute (e.g., trehalose) by fluid phase endocytosis to produce an internally loaded biological sample. The loading of a biological sample by fluid phase endocytosis comprises fusing within the biological sample a first matter (e.g., a vesicle) with a second matter (a lysosome) to produce a fused matter. The fused matter preferably comprises the solute. The loading of a biological sample by fluid phase endocytosis additionally comprises transferring the solute from the fused matter into a cytoplasm within the biological sample. The fused matter may comprise a lower pH than a pH of the first matter. The fused matter preferably comprises a pH of less than about 6.5. The biological sample may include a biological sample selected from a group of biological samples comprising a platelet and a cell.

Embodiments of the present invention also provide a process for preparing a dehydraded biological sample comprising providing a biological sample selected from a mammalian species, loading the biological sample with a solute by fluid phase endocytosis to produce a loaded biological sample, and drying the loaded biological sample to produce a dehydrated biological sample. The loading of the biological sample with a solute comprises loading of the biological sample with an oligosaccharide from an oligosaccharide solution, and preferably includes increasing a loading efficiency of the oligosaccharide into the biological sample by maintaining a concentration of the oligosaccharide in the oligosaccharide solution at less than a certain concentration (e.g., about 50 mM). The loading with an oligosaccharide includes loading with a loading efficiency ranging from about 45% to about 50% for the oligosaccharide solution having an oligosaccharide concentration ranging from about 20 mM to about 30 mM. The loading is preferably without a fixative. The process for preparing a dehydrated biological sample additionally comprises lyophilizing the biological sample, and prehydrating the lyophilized biological sample, preferably by exposing the lyophilized biological sample to moisture saturated air. When the biological sample comprises a platelet, and the process additionally comprises prehydrating the lyophilized platelet until the water content of the lyophilized platelet ranges from about 35% by weight to about 50% by weight.

Embodiments of the present invention also provide a process for loading a biological sample (e.g., a platelet and/or a cell) comprising loading by fluid phase endocytosis a biological sample with a solute (e.g., trehalose) and dimethylsulfoxide to produce an internally loaded biological sample. The loading of a biological sample by fluid phase endocytosis comprises fusing within the biological sample a first matter with a second matter (e.g., a lysosome) to produce a fused matter. Dimethylsulfoxide may be loaded into the cell by the same mechanism or by passive diffusion across the membrane. The first matter, as well as the fused matter,-comprises the solute and dimethylsulfoxide. The first matter may more specifically comprise a vesicle having the solute and dimethylsulfoxide. Alternatively, dimethylsulfoxide that enters the cell by diffusion across the cell membrane will be free in the cytoplasm. The loading of a biological sample by fluid phase endocytosis may additionally comprise transferring the solute and dimethylsulfoxide from the fused matter, such as transferring into a cytoplasm within the biological sample. The fused matter comprises a lower pH than a pH of the first matter.

Embodiments of the present invention further also provide a process for preparing a dehydrated biological sample comprising providing a biological sample selected from a mammalian species (e.g., mesenchymal stem cells), loading the biological sample with a solute and dimethylsulfoxide to produce a loaded biological sample, and drying the loaded biological sample to produce a dehydrated biological sample. The loading of the biological sample with a solute and dimethylsulfoxide may include loading by fluid phase endocytosis of the biological sample with an oligosaccharide (e.g., trehalose) and dimethylsulfoxide from an oligosaccharide solution having the oligosaccharide and the dimethylsulfoxide. Alternatively, the dimethylsulfoxide may be loaded into the cell by diffusion through the cell membrane. The drying of the loaded biological sample may comprise drying (e.g., air drying) the biological sample until the loaded biological sample has a water content ranging from about 0.3 grams of water per gram of dry weight biological sample to about 2.7 grams of water per gram of dry weight biological sample. The oligosaccharide solution preferably comprises at least about 0.10 weight percent of dimethylsulfoxide.

Further embodiments of the present invention provide a process for increasing the survival of a biological sample comprising providing a biological sample, loading the biological sample with a carbohydrate and dimethylsulfoxide to produce a loaded biological sample, and drying the loaded biological sample while maintaining a residual water content in the biological sample of at least about 0.01 gram water per gram of dry weight of biological sample to increase survival of the biological sample. Drying may comprise drying the biological sample until the loaded biological sample has a water content ranging from about 0.3 grams of water per gram of dry weight biological sample to about 2.7 grams of water per gram of dry weight biological sample. The method may additionally comprise storing the dehydrated loaded biological sample to produce a stored biological sample, and rehydrating the stored biological sample.

Further embodiments of the present invention provide a process for improving intracellular distribution of a solute in a biological sample comprising loading a biological sample with a carbohydrate and dimethylsulfoxide to produce a loaded biological sample having improved intracellular distribution over the same biological sample having been loaded with the carbohydrate but without the dimethylsulfoxide. The biological sample may comprise a fraction selected from the group of fractions comprising a mitochondrial fraction, a lysosomal fraction, and mixtures thereof. The intracellular distribution of the solute is improved in the fraction.

Additional embodiments of the present invention provide a method for increasing the survival of a biological sample (e.g., a mesenchymal stem cell) comprising loading a biological sample with a carbohydrate to produce a loaded biological sample, and air drying the loaded biological sample while maintaining a residual water content in the biological sample of less than or equal to about 3.0 grams of water per gram of dry weight of biological sample to increase survival of the biological sample over the biological sample having been freeze-dried.

Embodiments of the present invention further also provide a solution for increasing the distribution of a solute in a biological sample. The solution comprises a solute, and at least about 0.10% by weight of dimethylsulfoxide. The solution may also comprise a suitable protein (e.g., BSA) and a suitable salt solution (e.g., PBS).

In another aspect of embodiments of the present invention, a hemostasis aid is provided where the above described freeze-dried platelets are carried on or by a biocompatible surface. A further component of the hemostasis, aid may be a therapeutic agent, such as an antibiotic, an antifungal, or a growth factor. The biocompatible surface may be a bandage or a thrombic surface, such as freeze-dried collagen. Such a hemostasis aid can be rehydrated just before the time of application, such as by hydrating the surface on or by which the platelets are carried, or, in case of an emergency, the dry hemostasis treatment aid could be applied directly to the wound or burn and hydrated in situ.

Methods of making and using various embodiments of the present invention are also described. One such method is a process of preparing a dehydrated composition comprising providing a source of platelets, effectively loading the platelets with trehalose to preserve biological properties, cooling the trehalose loaded-platelets to below their freezing point, and lyophilizing the cooled platelets. The trehalose loading includes incubating the platelets at a temperature from greater than about 25° C. to less than about 40° C. with a trehalose solution having up to about 50 mm trehalose therein. The process of using such a dehydrated composition-further may comprise rehydrating the platelets. The rehydration preferably includes a prehydration step wherein the freeze-dried platelets are exposed to warm, saturated air for a time sufficient to bring the water content of the freeze-dried platelets to between about 20 weight percent to about 35 weight percent.

In yet another aspect of embodiments of the present invention, a drug delivery composition is provided comprising platelets having a homogeneously distributed concentration of a therapeutic agent therein. The drug delivery composition is particularly useful for targeting the encapsulated drug to platelet-mediated sites.

Practice of embodiments of the present invention permits the manipulation or modification of platelets while maintaining, or preserving, biological properties, such as a response to thrombin. Further, use of the method to preserve platelets can be practiced on a large, commercially feasible scale, and avoids platelet activation. Embodiments of the freeze-dried platelets, and hemostasis aids including the freeze-dried platelets, are substantially shelf stable at ambient temperatures when packaged in moisture barrier materials.

These provisions together with the various ancillary provisions and features which will become apparent to those skilled in the art as the following description proceeds, are attained by the processes and biological samples (e.g., platelets, eukaryotic cells, and erythrocytic cells) of the present invention, preferred embodiments thereof being shown with reference to the accompanying drawings, by way of example only, wherein:

### BRIEF DESCRIPTION OF THE DRAWINGS

FIG. 1 graphically illustrates the loading efficiency of trehalose plotted versus incubation temperature of human platelets.

FIG. 2 graphically illustrates the loading efficiency (cytosolic concentration divided by the extracellular concentration, the sum multiplied by 100) following incubation as a function of incubation time.

FIG. 3 graphically illustrates the internal trehalose concentration of human platelets versus external trehalose concentration as a function of temperature at a constant incubation or loading time.

FIG. 4 graphically illustrates the loading efficiency of trehalose into human platelets as a function of external trehalose concentration.

FIG. 5 graphically illustrates the-recovery of platelet embodiments after lyophilization and direct rehydration with various concentrations of trehalose in the drying buffer, and in a combination of 30 mM trehalose and one percent HSA in the drying buffer.

FIG. 6 graphically illustrates the uptake of FITC dextran versus the external concentration compared with that of the marker, LYCH (with an incubation time of four hours).

FIG. 7 graphically illustrates the effect of prehydration on optical density of platelets.

FIG. 8 illustrates the response of 500 μl platelets solution (with a platelet concentration of 0.5×10⁸ cells/ml) that was transferred to aggregation vials, thrombin added (1 U/ml) to each sample, and the samples stirred for three minutes at 37° C., where panel (A) are the prior art platelets and panel (B) are the inventive platelets.

FIG. 9 graphically illustrates clot formation where the absorbance falls sharply upon addition of thrombin (1 U/ml) and the platelet concentration drops from 250×10⁶ platelets/ml to below 2×10⁶ platelets/ml after three minutes for the inventive platelets.

FIG. 10 is an exemplary diagram of a biological sample having a plasma membrane with an internal protein coating and encapsulating a cytoplasm having lysosomes and a nucleus.

FIG. 11 is an elevational view of the plasma membrane in contact with a solute solution having a solute which is to be loaded into the biological sample.

FIG. 12 is an elevational view of the plasma membrane in the process of-being loaded with a solute.

FIG. 13 is an elevational view of a vesicle containing a solute and connected to the plasma membrane.

FIG. 14 is a diagram of the cytoplasm having a lysosome and a vesicle containing a solute and which “budded off” or released from the plasma membrane.

FIG. 15 is a diagram of a lysosome fused with a vesicle to produce fused matter or material containing a solute.

FIG. 16 is a diagram of the fused matter or material containing a solute which is in the process of passing in direction of the arrow from the fused matter or material into the cytoplasm of the biological sample to effectively load the biological sample with the solute.

FIG. 17 is an enlarged chemical structural, chain formula diagram of trehalose, a non-reducing disaccharide of glucose, with an arrow pointing to a glycosidic bond.

FIG. 18 is an enlarged chemical structural, chain formula diagram of sucrose, a non-reducing disaccharide of glucose and fructose, with an arrow pointing to a glycosidic bond which is much more susceptible to hydrolysis than the glycosidic bond in trehalose.

FIG. 19 is a graph of pH vs. % intact (i.e., % non-degraded) for trehalose and sucrose.

FIG. 20 is a graph of % leakage of a fluorescent dye, carboxyfluorescein (CF), from phospholipid vesicles as a function of pH and time.

FIG. 21 is a graph of rates of leakage (% leakage/10 minutes) as a function of pH.

FIG. 22 is a graph of projected time to achieve 100% leakage, based on FIGS. 20 and 21, as a function of pH.

FIG. 23 is a picture of control cells at zero (0) incubations time, showing no leakage of Lucifer yellow dye into the cytoplasm of the control cell.

FIG. 24 is a picture of cells after 1 hour incubation time, showing Lucifer yellow dye in punctate structures (i.e., endocytotic vesicles) with some leakage of Lucifer yellow dye into the cytoplasm.

FIG. 25 is a picture of cells after 3.5 hours incubation time, showing Lucifer yellow dye in punctuated structures (i.e., endocytotic vesicles) with more leakage of Lucifer yellow dye into the cytoplasm than the leakage represented in the picture of FIG. 24.

FIG. 26 is a picture of cells after 5.0 hours incubation time, showing a uniform stain of Lucifer yellow dye which suggests that Lucifer yellow dye has leaked into the cytoplasm.

FIG. 27 is a graph of survival (% viability) vs. water content (gm. water/gm. dry weight) for a first batch of mesenchymal stem cells (MSC cells) after air drying and rehydration, and for a second batch of mesenchymal stem cells MSC cells) after freeze drying and rehydration, with both batches of the mesenchymal stem cells (MSC cells) having trehalose internally.

FIG. 28 is a picture of control MSC cells at five hours of incubation time in an incubation solution having no DMSO present, with the LYCH fluorescence seen predominantly within endosomes as indicated by the punctate staining.

FIG. 29 is a picture of MSC cells at five hours of incubation time in an incubation solution having 2% by weight DMSO present for final 30 minutes of incubation, with slightly more LYCH fluorescence diffuse staining in the cytoplasm being seen over the staining seen in FIG. 28.

FIG. 30 is a picture of MSC cells at five hours of incubation time in an incubation solution having 2% by weight DMSO present for the entire five hours of incubation, with LYCH fluorescence diffuse staining being seen throughout the cytoplasm, indicating that DMSO provides benefit to the MSC cells by aiding the release of solutes from the endosomes and allowing a more homogeneious intracellular distribution.

FIG. 31 is a graph of total trehalose (% total trehalose) vs. cell fractionation (i.e., unbroken cells(N), mitochondrial fraction (M), and a lysosomal fraction (L)) after trehalose loading with and without DMSO.

FIG. 32 is a graph of survival (% viability) vs. water content (gm. water/gm. dry weight) for a first batch of mesenchymal stem cells (MSC cells) loaded with trehalose and after air drying and rehydration, and for a second batch of mesenchymal stem cells (MSC cells) loaded with trehalose and DMSO and after air drying and rehydration.

FIG. 33 is a graph of survival (% viability) vs. water content (gm. water/gm. dry weight) produced from the experiment of Example 15 for a first batch of mesenchymal stem cells (MSC cells) loaded with trehalose and after vacuum drying and rehydration, and for a second batch of mesenchymal stem cells MSC cells) loaded with trehalose and DMSO (at the end of the incubation period) and after vacuum drying and rehydration, reflecting that DMSO improves viability following vacuum-drying.

### DETAILED DESCRIPTION OF PREFERRED EMBODIMENTS OF THE INVENTION

Embodiments of the present invention broadly include biological samples, preferably mammalian biological samples. Embodiments of the present invention further broadly include methods for preserving biological samples, as well as biological samples that have been manipulated (e.g., by drying to produce dehydrated biological samples) or modified (e.g., loaded with a chemical or drug) in accordance with methods of the present invention. Embodiments of the present invention also further broadly include methods for increasing the survival of biological samples, especially during drying and following drying, storing and rehydrating.

Biological samples for various embodiments of the present invention comprise any suitable biological sample, such as blood platelets and cells. The cells may be any type of cell including, not by way of limitation, erythrocytic cells, eukaryotic cells or any other cell, whether nucleated or non-nucleated.

The term “erythrocytic cell” is used to mean any red blood cell. Mammalian, particularly human, erythrocytes are preferred. Suitable mammalian species for providing erythrocytic cells include by way of example only, not only human, but also equine, canine, feline, or endangered species.

The term “eukaryotic cell” is used to mean any nucleated cell, i.e., a cell that possesses a nucleus surrounded by a nuclear membrane, as well as any cell that is derived by terminal differentiation from a nucleated cell, even though the derived cell is not nucleated. Examples of the latter are terminally differentiated human red blood cells. Mammalian, and particularly human, eukaryotes are preferred. Suitable mammalian species include by way of example only, not only human, but also equine, canine, feline, or endangered species.

The-source of the eukaryotic cells may be any suitable source such that the eukaryotic cells may be cultivated in accordance with well known procedures, such as incubating the eukaryotic cells with a suitable serum (e.g., fetal bovine serum). After the eukaryotic cells are cultured, they are subsequently harvested by any conventional procedure, such as by trypsinization, in order to be loaded with a protective preservative. The eukaryotic cells are preferably loaded by growing the eukaryotic cells in a liquid tissue culture medium. The preservative (e.g., an oligosaccharide, such as trehalose) is preferably dissolved in the liquid tissue culture medium, which includes any liquid solution capable of preserving living cells and tissue. Many types of mammalian tissue culture media are known in the literature and available from commercial suppliers, such as Sigma Chemical Company, St. Louis, Mo., USA: Aldrich Chemical Company, Inc., Milwaukee, Wis., USA; and Gibco BRL Life Technologies, Inc., Grand Island, N.Y., USA. Examples of media that are commercially available are Basal Medium Eagle, CRCM-30 Medium, CMRL Medium-1066, Dulbecco's Modified Eagle's Medium, Fischer's Medium, Glasgow Minimum Essential Medium, Ham's F-10 Medium, Ham's F-12 Medium, High Cell Density Medium, Iscove's Modified Dulbecco's Medium, Leibovitz's L-15 Medium, McCoy's 5A Medium (modified), Medium 199, Minimum Essential Medium Eagle, Alpha Minimum Essential Medium, Earle's Minimum Essential Medium, Medium NCTC 109, Medium NCTC 135, RPMMI-1640 Medium, William's Medium E, Waymouth's MB 752/1 Medium, and Waymouth's MB 705/1 Medium.

Broadly, the preparation of solute-loaded biological sample(s) (e.g., platelets and cells) in accordance with embodiments of the invention comprises the steps of loading one or more biological samples with a solute by placing the biological samples in a solute solution for transferring by fluid phase endocytosis the solute from the solution into the biological sample(s). For increasing the transfer or uptake of the solute from the solute solution, the solute solution temperature, or incubation temperature, may have a temperature above about 25° C., more preferably above 30° C., such as from about 30° C. to about 40° C.

The method may additionally comprise preventing a decrease in a loading gradient and/or a loading efficiency gradient in the loading of the solute into the biological sample(s). Preventing a decrease in a loading efficiency gradient in the loading of the solute into the biological sample(s) comprises maintaining a positive gradient of loading efficiency (e.g., in %) to concentration (e.g., in mM) of the solute in the solute solution. Preventing a decrease in a loading gradient in the loading of the solute into the biological sample(s) comprises maintaining a concentration of the solute in the solute solution below a certain concentration (e.g., below a concentration ranging from about 35 mM to about 65 mM, more particularly below from about 40 mM to about 60 mM, or below from about 45 mM to about 55 mM, such as below about 50 mM); and/or maintaining a positive gradient of concentration of solute loaded into the biological sample(s) to concentration of the solute in the solute solution.

The solute solution may be any suitable physiologically acceptable solution in an amount and under conditions effective to cause uptake or “introduction” of the solute from the solute solution into the biological sample(s) for fluid phase endocytosis. A physiologically acceptable solution is a suitable solute-loading buffer, such as any of the buffers stated in the previously mentioned related patent applications, all having been incorporated herein by reference thereto.

The solute is preferably a carbohydrate (e.g., an oligosaacharide) selected from the following groups of carbohydrates: a monosaccharide, an oligosaccharide (e.g., bioses, trioses, tetroses, pentoses, hexoses, heptoses, etc), a disaccharide (e.g., lactose, maltose, sucrose, melibiose, trehalose, etc), a trisaccharide (e.g., raffinose, melezitose, etc), or tetrasaccharides (e.g., lupeose, stachyose, etc), and a polysaccharide (e.g., dextrins, starch groups, cellulose groups, etc). More preferably, the carbohydrate is a disaccharide, with trehalose being the preferred, particularly since it has been discovered that trehalose does not degrade or reduce in complexity upon being loaded. Thus, in the practice of various embodiments of the invention, trehalose is transferred from a solution into the biological sample without degradation of the trehalose. In other embodiments of the present invention, the solute may be dimethylsulfoxide (DMSO) alone, or a combination of an oligosaacharide (e.g., trehalose) and DMSO.

Compositions and embodiments of the invention include platelets that have been manipulated (e.g. by freeze-drying) or modified (e.g. loaded with drugs), and that are useful for therapeutic applications, particularly for platelet transfusion therapy, as surgical or hemostasis aids, such as wound dressings, bandages, and as sutures, and as drug-delivery vehicles. As has been known, human platelets have a phase transition between 12° C. and 20° C. We have found that platelets have a second phase transition between 30° C. and 37° C. Our discovery of this second phase transition temperature range suggests the possible use of platelets as vehicles for drug delivery because we can load platelets with various useful therapeutic agents without causing abnormalities that interfere with normal platelet responses due to changes, such as in the platelet outer membranes.

For example, platelets may be loaded with anti-thrombic drugs, such as tissue plasminogen activator (TPA) so that the platelets will collect at the site of a thrombus, as in an heart attack, and release the “clot busting” drug or drugs that are encapsulated and have been targeted by the platelets. Antibiotics can also be encapsulated by the platelets, since lipopolysaccharides produced by bacteria attract platelets. Antibiotic loaded platelets will bring the selected antibiotics to the site of inflammation. Other drugs that can be loaded include anti mitotic agents and anti-angiogenic agents. Since platelets circulate in newly formed vessels associated with tumors, they could deliver anti-mitotic drugs in a localized fashion, and likely platelets circulating in the neovasculature of tumors can deposit anti-angiogenic drugs so as to block the blood supply to tumors. Thus, platelets loaded with a selected drug in accordance with this invention can be prepared and used for therapeutic applications. The drug-loaded platelets are particularly contemplated for blood-borne drug delivery, such as where the selected drug is targeted to a site of platelet-mediated forming thrombi or vascular injury. The so-loaded platelets have a normal response to at least one agonist, particularly to thrombin. Such platelets can be loaded additionally with trehalose, if preservation by freeze-drying is intended.

The key component for compositions and apparatus of embodiments of the invention, when preservation will be by freeze-drying, is a lyoprotectant, preferably an oligosaccharide, more preferably trehalose, because we have found that platelets that are effectively loaded with trehalose preserve biological properties during freeze-drying (and rehydration). This preservation of biological properties, such as the normal clotting response in combination with thrombin, is necessary so that the platelets following preservation can be successfully used in a variety of therapeutic applications.

Normal hemostasis is a sequence of interactions in which blood platelets contribute, beginning with adhesion of platelets to an injured vessel wall. The platelets form an aggregate that accelerates coagulation. A complex, termed the glycoprotein (GP) 1b-IX-V complex, is involved in platelet activation by providing a binding site on the platelet surface for the potent agonist, (-thrombin. a-thrombin is a serine protease that is released from damaged tissue. Thus, it is important that the manipulations and modifications in accordance with this invention do not activate the platelets. Further, it is normally preferred that the platelets be in a resting state. Otherwise, the platelets will activate.

Although for most contemplated therapeutic applications the clotting response to thrombin is key, the inventive freeze-dried platelets after rehydration will also respond to other agonists besides thrombin. These include collagen, ristocetin, and ADP (adenosine diphosphate), all of which are normal platelet agonists. These other agonists typically pertain to specific receptors on the platelet's surface.

Broadly, the preparation of preserved platelets in accordance with the invention comprises the steps of providing a source of platelets, loading the platelets with a protective oligosaccharide at a temperature above about 25° C. and less than about 40° C., cooling the loaded platelets to below −32° C., and lyophilizing the platelets.

In order to provide a source of platelets suitable for the inventive preservation process, the platelets are preferably isolated from whole blood. Thus, platelets used in this invention preferably have had other blood components (erythrocytes and leukocytes) removed prior to freeze-drying. The removal of other blood components may be by procedures well known to the art, which typically involve a centrifugation step.

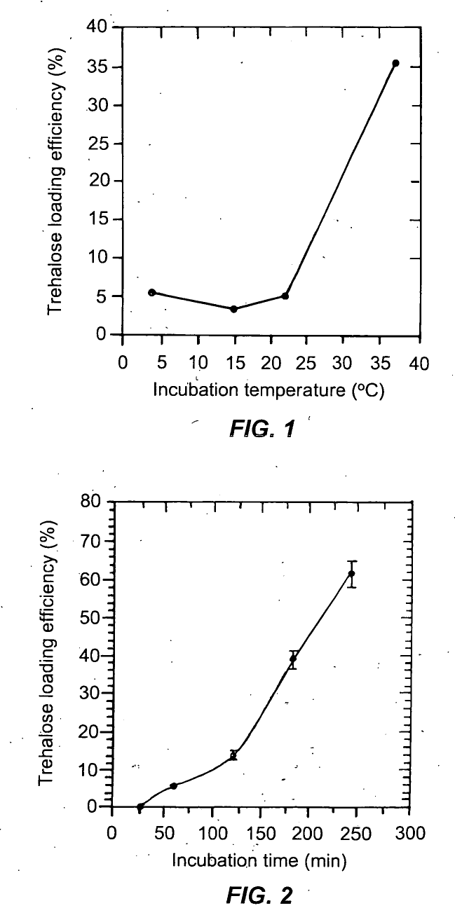

The amount of the preferred trehalose loaded inside the inventive platelets is from about 10 mM to about 50 mM, and is achieved by incubating the platelets to preserve biological properties during freeze-drying with a trehalose solution that has up to about 50 mM trehalose therein. Higher concentrations of trehalose during incubation are not preferred, as will be more fully explained later. The effective loading of trehalose is also accomplished by means of using an elevated temperature of from greater than about 25° C. to less than about 40° C., more preferably from about 30° C. to less than about 40° C., most preferably about 37° C. This is due to the discovery of the second phase transition for platelets. As can be seen by FIG. 1, the trehalose loading efficiency begins a steep slope increase at incubation temperatures above about 25° C. up to about 40° C. The trehalose concentration in the exterior solution (that is, the loading buffer) and the temperature during incubation together lead to a trehalose uptake occurring primarily through fluid phase endocytosis. FIG. 2 illustrates the trehalose loading efficiency as a function of incubation time.

As indicated in patent application Ser. No. 10/052,162, which claims the benefit of patent application Ser. No. 09/501,773, filed Feb. 10, 2000, with respect to common subject matter, the amount of the preferred trehalose loaded inside the cells ranges from about 10 mM to about 50 mM, and is achieved by incubating the cells to preserve biological properties during freeze-drying with a trehalose solution, preferably a trehalose solution that has up to about 50 mM trehalose therein. Higher concentrations of trehalose during incubation are not preferred, particularly since an embodiment of the invention includes preventing a decrease in a loading gradient, or a loading efficiency gradient, in the loading of the solute into the cell. It has been discovered that preventing a decrease in a loading gradient, or a loading efficiency gradient, in the loading of a oligosaccharide (i.e., trehalose) into a cell comprises maintaining a concentration of the oligosaccharide in the oligosaccharide solution below a certain concentration (e.g., below a concentration ranging from about 35 mM to about 65 mM, more particularly below from about 40 mM to about 60 mM, or below from about 45 mM to about 55 mM, such as below about 50 mM). It has been further discovered that preventing a decrease in a loading gradient, or a loading efficiency gradient, in the loading of a oligosaccharide (i.e., trehalose) into a cell comprises maintaining a positive gradient of loading efficiency to concentration of the oligosaccharide in the oligosaccharide solution.

As further indicated in co-pending patent application Ser. No. 10/052,162, the effective loading of trehalose is also accomplished by means of using an elevated temperature of from greater than about 25° C. to less than about 40° C., more preferably from about 30° C. to less than about 40° C., most preferably about 37° C. This is due to the discovery of the second phase transition for cells.

Referring now to FIG. 1, there is seen a graphical illustration from co-pending patent application Ser. No. 10/052,162 of the loading efficiency of trehalose plotted versus incubation temperature of human platelets. The trehalose loading efficiency begins a steep slope increase at incubation temperatures above about 25° C. and continues up to about 40° C. The trehalose concentration in the exterior solution (that is, the solute solution or loading buffer) and the temperature during incubation together lead to a trehalose uptake that occurs through fluid phase endocytosis. Example 1 below provides the more specific testing conditions and parameters which produced the graphical illustrations of FIG. 1. It is believed that the graphical illustration of the loading efficiency in FIG. 1 would be generally applicable for cells in general.

Referring now to FIG. 2, there is seen an illustration from co-pending patent application Ser. No. 10/052,162 of trehalose loading efficiency for human blood platelets as a function of incubation time. More specifically, FIG. 2 graphically illustrates the loading efficiency (cytosolic concentration divided by the extracellular concentration, the sum multiplied by 100) following incubation as a function of incubation time. Example 1 below provides the more specific testing conditions and parameters which produced the graphical illustrations of FIG. 2. It is believed that the graphical illustration of the loading efficiency in FIG. 2 would also be generally applicable for cells in general.

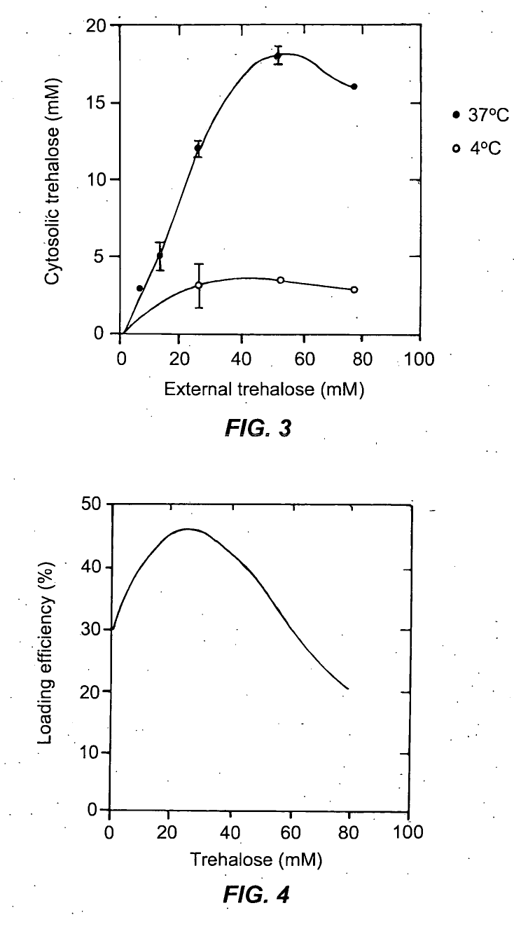

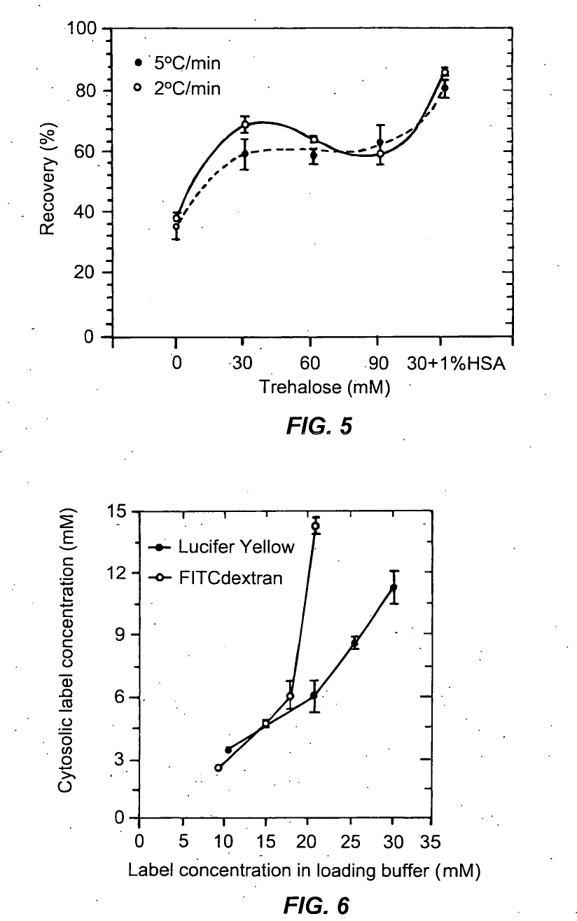

Referring now to FIG. 3, there is seen a graphical illustration from patent application Ser. No. 10/052,162 of the internal trehalose concentration of human platelets versus external trehalose concentration as a function of 4° C. and 37° C. temperatures at a constant incubation or loading time. In FIG. 4 there is seen a graphical illustration from patent application Ser. No. 10/052,162 of the loading efficiency of trehalose into human platelets as a function of external trehalose concentration. Example 1 below provides the more specific testing conditions and parameters which produced the graphical illustrations of FIGS. 3 and 4. In additional embodiments of the present invention, it is further believed that the general findings illustrated in FIGS. 3 and 4 with respect to platelets are generally broadly applicable to cells in general.

Thus, applying the findings illustrated in FIG. 3 and in FIG. 4 to solutes and cells in general, a decrease in a loading gradient or a loading efficiency gradient in the loading of a solute into a cell may be prevented. For an embodiment of the present invention and as broadly illustrated in FIG. 3, preventing a decrease in a loading gradient or a loading efficiency gradient in the loading of the solute (e.g., an oligosaccharide such as trehalose) into the cell comprises maintaining a concentration of the solute (e.g., an oligosaccharide such as trehalose) in the solute solution (e.g. an oligosaccharide solution such as a trehalose solution) below a solute concentration ranging from about 35 mM to about 65 mM, more specifically a solute concentration ranging from about 40 mM to about 60 mM, more specifically further a solute concentration ranging from about 45 mM to about 55 mM (e.g., about 50 mM). In another embodiment of the present invention and as best illustrated in FIG. 4, preventing a decrease in a loading gradient or a loading efficiency gradient in the loading of the solute (e.g., an oligosaccharide, such as trehalose) into the cell comprises maintaining a positive gradient of loading efficiency (e.g., loading efficiency in %) to concentration (e.g., concentration in mM) of the solute in the solute solution (e.g. an oligosaccharide solution, such as a trehalose solution).

Loading of the solute from the solute solution broadly includes producing and/or forming at least a portion of the biological membrane to entrap and include a solute; and fusing, commingling, or otherwise combining in any suitable manner, the produced and/or formed solute-containing portion of the biological membrane with a lysosome to produce fused matter from which the solute is transferred into the cytoplasm of the biological membrane (e.g., a cell). Producing and/or forming at least a portion of the biological membrane to include the solute comprises transferring or passing the solute from the solute solution against and/or into a portion of the biological membrane for producing and/or forming a vesicle (i.e., an endosomal, phagocytic vesicle) containing the solute. The vesicle subsequently breaks or severs (i.e., “buds off”) from the biological membrane into the cytoplasm of the biological sample(s) to fuse with lysosome(s).

The fusing or combining of the vesicle with a lysosome is caused by recognition sites on both membranes that promote fusion or the combining. The produced fused matter subsequently breaks down or degrades, with the lysosomal membranes being recycled and reloaded in the Golgi. Most sugars are degraded in the lysosome to monosaccharides, which are then transferred to the cytoplasm for further degradation. It is suggested that the mechanism of transfer includes the magnitude of the internal pH in the lysosomes which leads to leakage across the bilayers. The internal, engulfed material within the fused matter contains a reduced pH (e.g., a pH ranging from about 3.5 to about 6.0). In additon there is the presence of acidic-hydrolases in the lysosomes.

The reduced pH, an acidic pH, causes the membrane of the produced fused matter to have an increased permeability. Stated alternatively, lowering the pH of the internal, engulfed material through the fusing of lysosome and vesicles produces an acidic engulfed material within the fused matter, which concomitantly raises or increases the permeability of the membrane of the fused matter. With an increase in permeability, the solute (or any low molecular weight molecules) leaks or passes through the membrane of the fused matter and into the cytoplasm.

When the solute is a sugar, most sugars hydrolyze within the fused matter. An exception is trehalose, which escapes degradation due to the stability of its associated glycosidic linkage. The broken down components of the lysosome and the vesicles are released into the cytoplasm for further metabolism. The components of sucrose would include glycose and fructose, which are degraded by the well known glycolytic pathway and the TCA cycle to CO₂ and H₂O. Because trehalose remains in tact for effecting the transferring and the loading of the solute into the cytoplasm of the biological sample(s), and does not degrade in conditions found in the lysome-endosome, trehalose is a preferred solute. However, it is to be understood that while trehalose is a preferred solute, the spirit and scope of the present invention includes any solute comprising one or more molecules that survive the environmental conditions within the fused matter. More specifically, the solute for various embodiments of the present invention comprises one or more of any molecule(s) that does not degrade under the transferring or loading conditions, or within the environmental conditions within the fused matter resulting from the fusing of lysosome and the vesicle. After the solute is transferred out of the fused matter and into the cytoplasm, stability is conferred on the biological sample for further treatment or processing, such as drying.

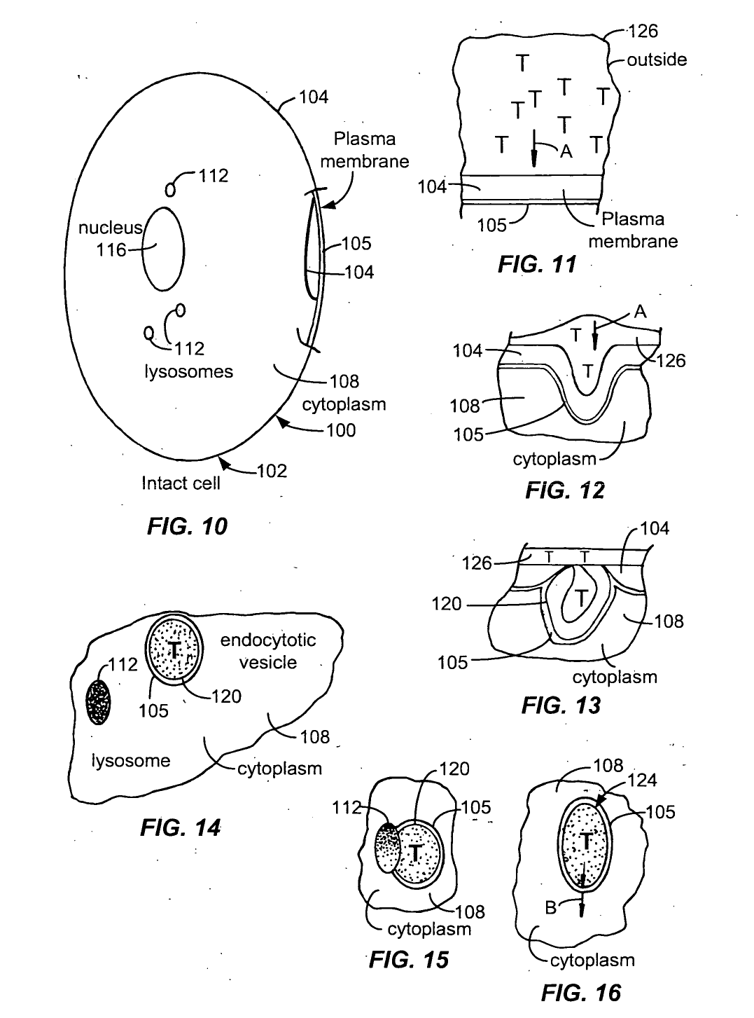

Referring now to FIGS. 10-16 for more specifically describing an embodiment of a mechanism for loading by fluid phase endocytosis a solute from a solute solution into a biological sample (e.g., platelet(s), cell(s), etc.), there is seen in FIG. 10 a biological sample 100 which is exemplarily represented as an intact cell 102 having a plasma membrane 104 internally coated with a protein (e.g., clathrin) 105. The plasma membrane 104 encapsulates cytoplasm 108 having lysosomes 112. The plasma membrane 104 may also encapsulate a nucleus 116 contained within the cytoplasm 108.

The biological sample 100 is disposed in a solute solution 126 having a solute T (e.g., trehalose). As shown in FIG. 11, the solute T is transferred or passed in direction of the arrow A from the solute solution 126 against and/or into a portion of the membrane 104. As previously indicated, the solute solution 126 may be heated to an elevated temperature (e.g., a temperature from about 30° C. to about 40° C.) to assist in transferring the solute T out of the solute solution 126 and against and/or into a portion of the membrane 104, causing the plasma membrane 104 including its associated protein coat 105 to bulge and/or concave inwardly (as best shown in FIG. 12) to begin the formation of a portion of the membrane 104 having the solute T; that is, a vesicle 120 (see FIG. 13) begins to form. Referring now to FIG. 14 these is seen a partial plan view of the biological sample 100 after the subsequent release or “budding off” of the vesicle 120 into the cytoplasm 108. The vesicle 120 is coated with the protein 105 and contains the solute T. As exemplarily shown in FIG. 15, the vesicle 120 fuses with lysosome 112 to produce and/or form fused matter 124 which is also coated with the protein 105.

The internal, engulfed material within the fused matter 124 contains a reduced pH (e.g., a pH ranging from about 3.5 to about 6.0) due to ion pumps in the membrane. The acid hydrolases are activated by the low pH. The reduced pH of the internal, engulfed material causes the outer skin or membrane of the produced fused matter 124 to have an increased permeability which facilitates the leakage or passage of the solute (or any low molecular weight molecules) through the outer skin or membrane of the fused matter 124, as illustrated in FIG. 16. As previously indicated, when the solute is trehalose or any other low molecular weight molecule that is immune to the acidic engulfed material within the fused matter 124, trehalose escapes degradation due to the stability of its associated glycosidal linkage and freely passess in tact through the increased-permeability membrane of the fused matter. As previously suggested, the remaining broken down components of the lysosome and the vesicle are released into the cytoplasm for further metabolism. Thus, the solute T is transferred out of the fused matter 124, as represented by arrow B in FIG. 16, when the permeability of the membrane of the fussed matter 124 is increased, and when the engulfed material within the fused matter 124 breaks down or degrades for further metabolism within the cytoplasm. As previously indicated, the solute T preferably remains intact during the loading and/or solute transferring process and within the internal environment of the fused matter 124. Thus, the solute T remains essentially intact and whole when transferred out of the fused matter 124 and into the cytoplasm 108. The solute T survives conditions found in the lysosome-endosome and the intact solute T leaks through the outer membrane of the fused matter 124 and into the cytoplasm. The biological sample 100 is now ready for further processing, such as drying, freezing, and subsequent rehydration, etc.

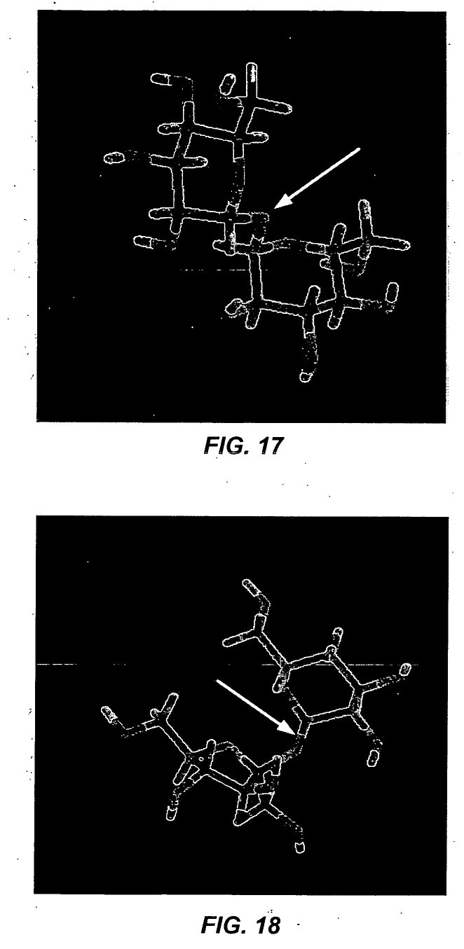

A preferred solute for embodiments of the present invention comprises trehalose. Most sugars degrade in fused lysosome-endosome due to the reduced pH and presence of acid hydrolases. Trehalose is the only non-reducing disaccharide of glusose. FIG. 17 is an enlarged chemical structural, chain formula diagram of trehalose, a non-reducing disaccharide of glucose, with an arrow pointing to a glycosidic bond. Severing of the glycosidic bond produces glucose which is ineffective in stabilizing dry biological materials. Sucrose, on the other hand, is a non-reducing disaccharide of glucose and fructose. FIG. 18 is an enlarged chemical structural, chain formula diagram of sucrose, a non-reducing disaccharide of glucose and fructose, with an arrow pointing to a glycosidic bond which is much more susceptible to hydrolysis than the glycosidic bond in trehalose. Trehalose survives conditions found in the lysosome-endosome and intact trehalose leaks into the cytosol of living cells.

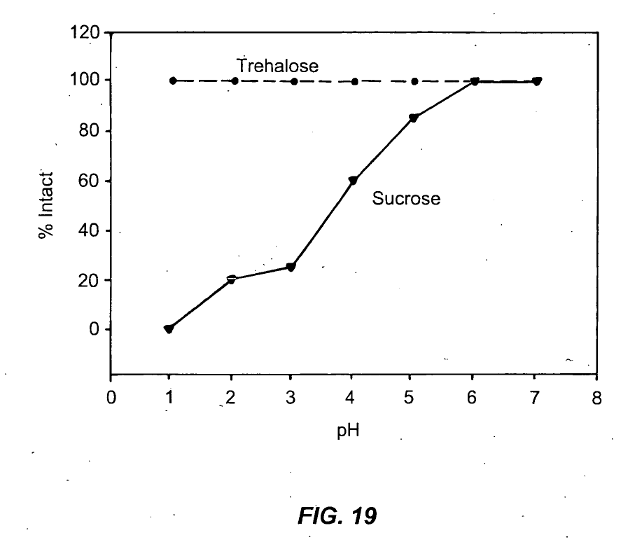

Referring now to FIG. 19, there is seen a graph of pH vs. % intact (i.e., % non-degraded) for trehalose and sucrose. Trehalose survives survival (i.e., remains 100% intact) down to a pH 1, while sucrose hydrolyzes into glucose and. fructose at pH as 5. The % of intact sucrose commences to decrease below a pH of about 6. Thus, sucrose begins to break down at a pH below 6. Example 7 below provides the more specific testing conditions and parameters which produced the graphical, illustrations of FIG. 19.

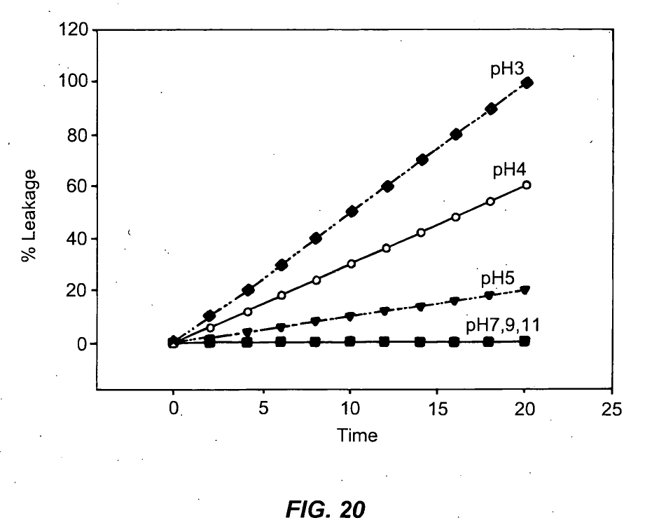

FIG. 20 is a graph of % leakage of a fluorescent dye, carboxyfluorescein (CF), from phospholipid vesicles as a function of pH and time. As the pH decreases from about 7.0 to a pH of about 3.0 and as time increases (e.g., increases from about 0 to about 20 minutes, the % leakage of the fluorescent dye increases. There is little or no leakage at a pH of about 7.0 or above, but leakage proceeds rapidly at a pH below about 5.0. At pH of about 3.0, 100% of the solute leaked out in 20 minutes. Thus, the leakage of the fluorescent dye CF from liposomes increases with pH and time.

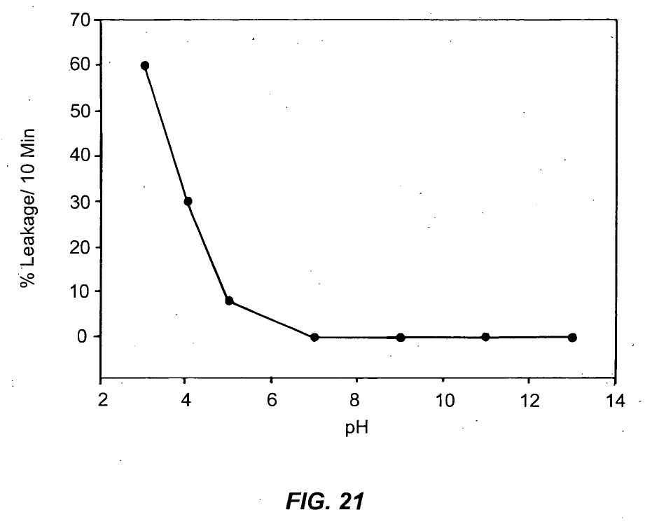

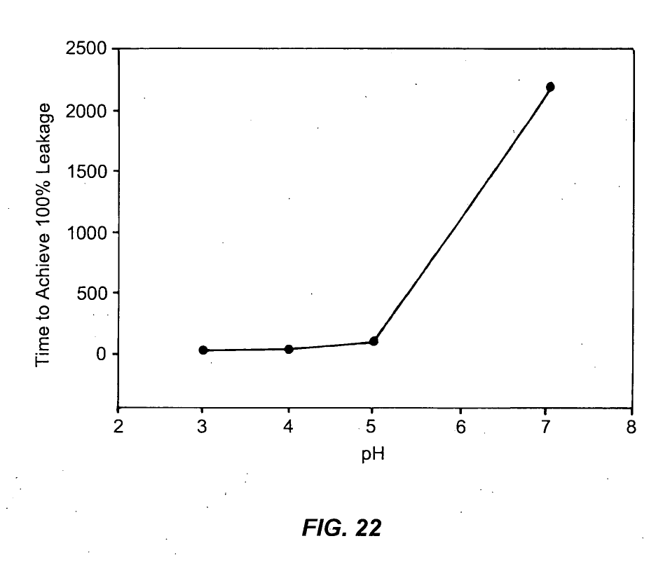

With respect to rate of leakage and the time for leakage, the rate of leakage increases as the pH decreases, as best illustrated in FIG. 21, and the time to achieve 100% leak increases with increase in pH, as best shown in FIG. 22. FIG. 21 is a graph of rates of leakage (% leakage/10 minutes) as a function of pH. At pH of 3-4 leakage goes to completion in 20-30 minutes, while at pH 7, three months would be required to complete the leakage. FIG. 22 is a graph of projected time to achieve 100% leakage, based on FIGS. 20 and 21, as a function of pH. The time to achieve 100% depletion especially increases after a pH of 5. Example 8 below provides the more specific testing conditions and parameters which produced the graphical, illustrations of FIGS. 20-22.

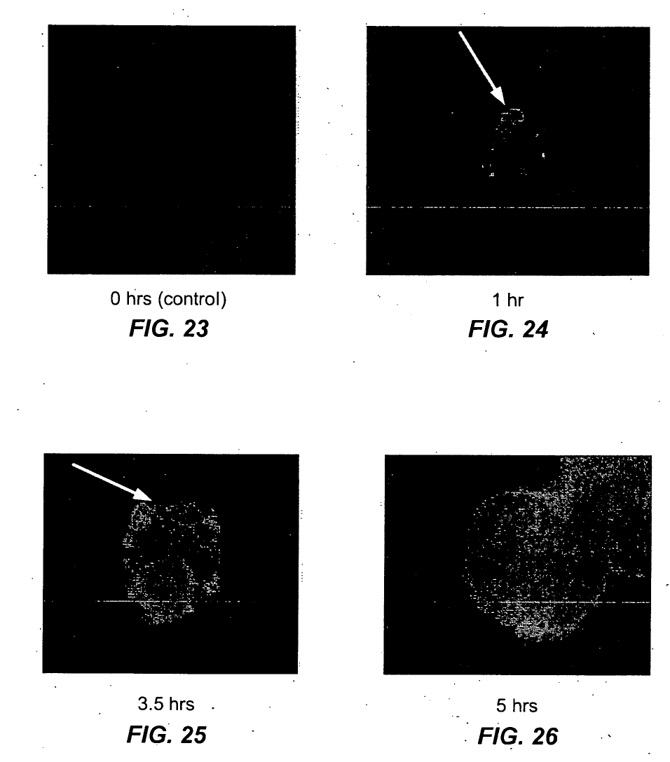

Referring now to FIGS. 23-26, there is seen a distribution of Lucifer yellow in intact cells as a function of incubation time. More specifically, FIG. 23 is a picture of control cells at zero (0) incubation time, showing no leakage of Lucifer yellow dye into the cytoplasm of the control cell. FIG. 24 is a picture of cells after 1 hour incubation time, showing Lucifer yellow dye in punctate structures (i.e., endocytotic vesicles) with some leakage of Lucifer yellow dye into the cytoplasm. FIG. 25 is a picture of cells after 3.5 hours incubation time, showing Lucifer yellow dye in punctate structures (i.e., endocytotic vesicles) with more leakage of Lucifer yellow dye into the cytoplasm than the leakage represented in the picture of FIG. 24; and FIG. 26 is a picture of cells after 5.0 hours incubation time, showing a uniform stain of Lucifer yellow dye which suggests that Lucifer yellow dye has leaked into the cytoplasm. At short incubation times (e.g., incubation times of 1 hour and 3.5 hours), the dye is in punctate structures. With long incubation time (e.g., 5 hours) the staining becomes uniform, suggesting that the dye has leaked into the cytoplasm. Example 9 below provides the more specific testing conditions and parameters which produced the graphical, illustrations of FIGS. 23-26.

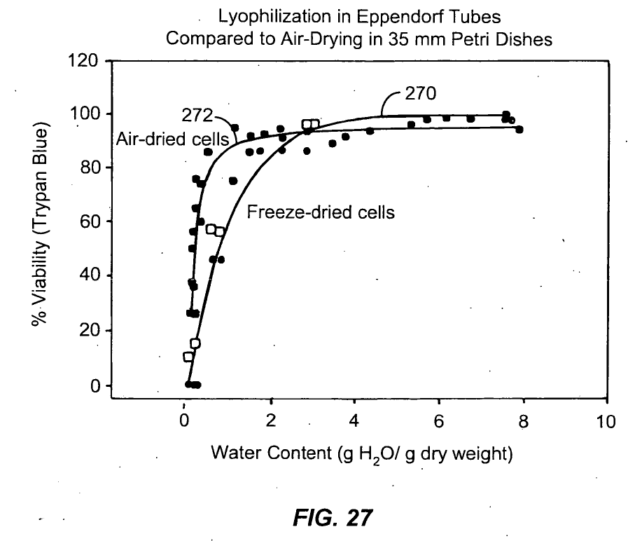

Referring in detail now to FIGS. 27-32 for further embodiments of the invention, there is seen in FIG. 27 a graph of survival (% viability) vs. water content (gm. water/gm. dry weight) for a first batch of mesenchymal stem cells (MSC cells) after air drying and rehydration, and for a second batch of mesenchymal stem cells (MSC cells) after freeze drying and rehydration, with both batches of the mesenchymal stem cells (MSC cells) having trehalose internally. Example 11 below provides the more specific testing conditions and parameters which produced the graphical illustrations of FIG. 27. Broadly for Example 11, mesenchymal stem cells were loaded with trehalose for 24 hours by incubation at 37° C. in medium+100 mM trahalose. The cells were either lyophilized in Eppendorf tubes on a Virtis side-arm lyophilizer or air-dried (0.5 mL samples in 35 mm Petri dishes) in a sterile hood to various water contents. They were then rehydrated and viability assessed by trapan blue exclusion. It is clear that, below the critical water content of 2 gH₂O/g dry weight, the MSCs survived air-drying better than freeze drying.

Graph 270 and graph 272 in FIG. 27 represents freeze-dried mesenchymal stem cells, and air-dried mesenchymal stem cells, respectively. FIG. 27 broadly illustrates that cell survival increases (e.g., increases by from about 20% to about 90%) by air drying as opposed to freeze drying. FIG. 27 more specifically illustrates that after mesenchymal stem cells were loaded with trehalose (e.g., 25 mM to 800 mM trehalose) while incubating at a temperature above about 25° C. (e.g., from about 35° C. to about 40° C.), and then air dried, instead of or as opposed to freeze drying, until the mesenchymal stem cells comprised a residual water content of less than or equal to about 0.30 grams of water per-gram of dry weight of mesenchymal stem cells, survival (% viability) increases. FIG. 27 also more specifically illustrates that had the trehalose-loaded, mesenchymal stem cells been freeze dried, instead of or as opposed to air dried, to the extent that the trehalose-loaded, mesenchymal stem cells had a residual water content of greater than (or equal to) about 0.30 grams of water per gram of dry weight of mesenchymal stem cells, survival (% viability) increases. Thus, freeze drying is the preferred drying technique for trehalose-loaded, mesenchymal stem cells if the residual water content of the trehalose-loaded, mesenchymal stem cells is maintained at greater than (or equal to) about 0.30 grams of water per gram of dry weight of mesenchymal stem cells (e.g., from about 0.30 grams of water per gram of dry weight of mesenchymal stem cells to about 0.80 grams water per gram of dry weight of mesenchymal stem cells); and air drying is the preferred drying technique for trehalose-loaded, mesenchymal stem cells if the residual water content of the trehalose- loaded, mesenchymal stem cells is maintained at less than (or equal to) about 0.30 grams water per gram of dry weight of mesenchymal stem cells. As shown in FIG. 27, the survival of the mesenchymal stem cells (i.e., the biological sample) is preferably at least about 60% (e.g., such as from about 60% to about 90%), more preferably at least about 90%.

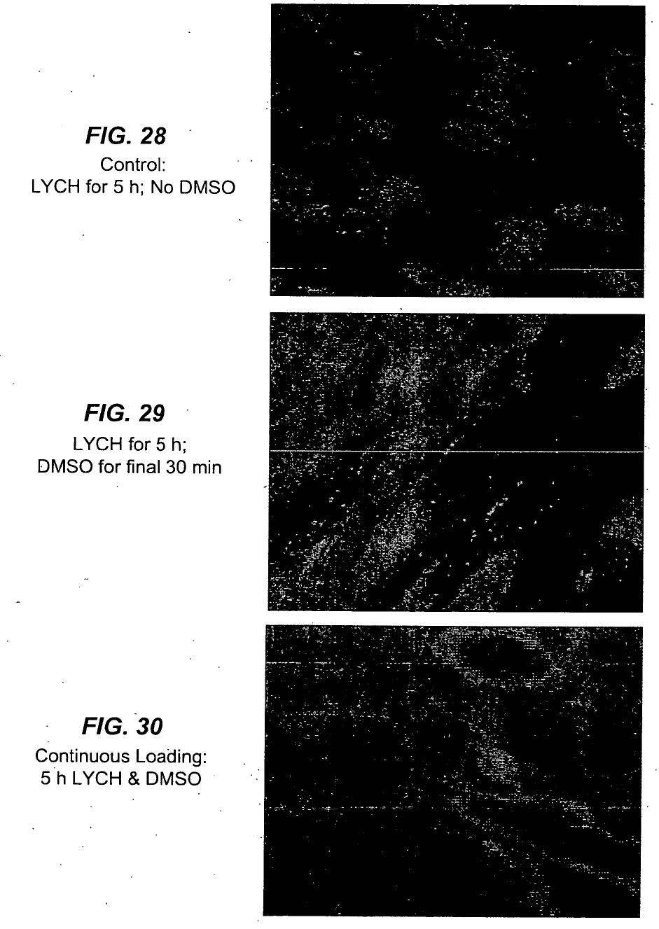

Referring now to FIGS. 28-30, there is seen a distribution of Lucifer yellow in MSC cells as a function of incubation solution having DMSO. More specifically, FIG. 28 is a picture of control MSC cells at five hours of incubation time in an incubation solution having no DMSO present, with the LYCH fluorescence seen predominantly within endosomes as indicated by the punctate staining. FIG. 29 is a picture of MSC cells at five hours of incubation time in an incubation solution having 2% by weight DMSO present for final 30 minutes of incubation, with slightly more LYCH fluorescence diffuse staining in the cytoplasm being seen over the staining seen in FIG. 28. FIG. 30 is a picture of MSC cells at five hours of incubation time in an incubation solution having 2% by weight DMSO present for the entire five hours of incubation, with LYCH fluorescence diffuse staining being seen throughout the cytoplasm, indicating that DMSO provides benefit to the MSC cells by aiding the release of solutes (e.g., trehalose) from the endosomes and allowing a more homogeneous intracellular distribution. Thus, by adding DMSO to a loading solution having trehalose, a more homogeneous distribution of trehalose in the biological sample (e.g., MSC cells) is provided. Example 12 below provides the more specific testing conditions and parameters which produced the graphical, illustrations of FIGS. 28-30.

The DMSO-containing solute solution for these embodiments of the present invention may be used for any suitable purpose, such as a loading or incubating solution, or as a drying solution, or a rehydrating solution. When the DMSO-containing solute solution is used for loading a solute, the solute solution would also comprise the solute, and optionally, a buffering-salt chemical or compound. The solute solution for these embodiments of the invention may be used for any biological sample, particularly for eukaryotic cells (i.e., MSC cells).

For embodiments of the invention where the DMSO-containing solute solution is used for loading a solute, the solute solution comprises at least about 1.0 weight % (e.g., at least about 25 mM) of a solute, at least about 0.5 weight % (e.g., at least about 60 mM) of dimethylsulfoxide (DMSO), optionally (with or without) at least about 1.0 weight % (e.g., at least about 0.1 mM) of a protein, and at least about 50.0 weight % of a salt solution. More specifically, where the DMSO-containing solute solution is used for loading a solute, the solute solution comprises from about 1 weight % to about 20 weight % (e.g., from about 25 mM to about 500 mM) of a solute (e.g., a starch, a carbohydrate, an oligosaacharide such as trehalose, etc.), from about 0.5 weight % to about 5 weight % (e.g., from about 60 mM to about 600 mM) of dimethylsulfoxide (DMSO), optionally (with or without) about 1 weight % to about 20 weight % (e.g., from about 0.15 mM to about 3.0 mM) of a protein (e.g., BSA), from about 50 weight % to about 99 weight % of a salt solution; more preferably from about 2 weight % to about 10 weight % (e.g., from about 50 mM to about 250 mM) of a solute (e.g., a starch, a carbohydrate, an oligosaacharide such as trehalose, etc.), from about 1 weight % to about 3 weight % (e.g., from about 125 mM to about 375 mM) of dimethylsulfoxide (DMSO), optionally (with or without) 2 weight % to about 10 weight % (e.g., from about 0.3 mM to about 1.5 mM) of a protein (e.g., BSA), from about 70 weight % to about 98 weight % of a salt solution; and most preferably from about 3 weight % to about 5 weight % (e.g., from about 80 mM to about 130 mM) of a solute (e.g., a starch, a carbohydrate, an oligosaacharide such as trehalose, etc.), from about 1.5 weight % to about 2.5 weight % (e.g., from about 20 mM to about 35 mM) of dimethylsulfoxide (DMSO), optionally (with or without) from about 3 weight % to about 8 weight % (e.g., from about 0.4 mM to about 1.2 mM) of a protein (e.g., BSA), from about 80 weight % to about 95 weight % of a salt solution.

The loading temperature of the DMSO-containing loading solution for loading DMSO and a solute (e.g., trehalose) protein into the biological sample(s) may be any suitable temperature, such as a temperature ranging from about 0 degrees C to about 60 degrees C., preferably from about 10 degrees C. to about 40 degrees C., more preferably from about 36 degrees C. to about 38 degrees C. The loading/incubating time for loading DMSO and the solute may be any suitable time, such as a time ranging from about 10 minutes to about 46 hours, preferably from about 30 minutes to about 40 hours, more preferably greater than about 6 hours, such as from about 6 hours to about 30 hours, most preferably from about 10 hours to about 24 hours. The time and temperature of incubation in the carbohydrate solution may be different from the incubation in the solution containing DMSO. For instance and by way of example only, for various embodiments of the invention the method may comprise incubating the cells in the carbohydrate solution for 21 hours at 37° C., at which time the DMSO is added and the incubation continued for 3 more hours, either at the same temperature or a lower temperature (˜20° C.).

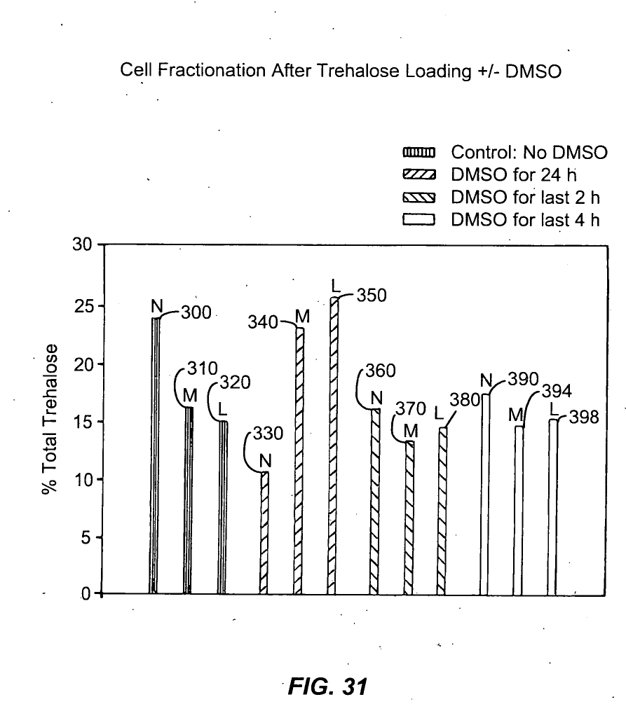

In other embodiments of the present invention, it has been discovered, as previously indicated, that DMSO aids in the intracellular distribution of a solute (e.g., trehalose) throughout a biological sample, as broadly illustrated by FIG. 31, which is a graph of total trehalose (% total trehalose) vs. cell fractionation (i.e., unbroken cells(N), mitochondrial fraction (M), and a lysosomal fraction (L)) after trehalose loading with and without DMSO. Example 13 below provides the more specific testing conditions and parameters which produced the graphical illustrations of FIG. 31. Broadly, Example 13 illustrates that DMSO improves the intracellular distribution of trehalose when included with the cells for the full 24 hour trehalose incubation. Mesenchymal stem cells were loaded with 100 mM trehalose for 24 hours at 37° C. DMSO (2%) was included in the incubation for the full 24 hours, for the last 2 hours, for the last 4 hours, or not at all (control). The cells were fractionated by differential centrifugation and separated into a nuclear fraction (which also includes unbroken cells:N), a mitochondrial fraction (M), and a lysosomal fraction (L). It can be seen that when DMSO is included in the full 24 hour incubation with trehalose (red bars), the mitochondrial and lysosomal fractions show increased trehalose concentrations as compared to the nuclear fraction, containing whole cells. Treating the samples with DMSO for just the last 2 or 4 hours of the trehalose incubation did not significantly change the trehalose concentrations of the M or L fractions compared to those of the control.

Graphs 300, 310 and 320 in FIG. 31, respectively, represent the distribution of solute (i.e., trehalose) in the following nuclear fractions of a biological sample (e.g., mesenchymal stem cells) after being loaded with 100 mM trehalose by incubating at 37° C. for 24 hours: unbroken cells(N), mitochondrial fraction (M), and a lysosomal fraction (L). Graphs 330, 340 and 350 in FIG. 31, respectively, represent the distribution of solute (i.e., trehalose) in the following nuclear fractions of a biological sample (e.g., mesenchymal stem cells) after being incubated at 37° C. for 24 hours in an incubation buffer having 2% by weight DMSO and 100 mM trehalose: unbroken cells(N), mitochondrial fraction (M), and a lysosomal fraction (L). Graphs 360, 370 and 380 in FIG. 31, respectively, represent the distribution of solute (i.e., trehalose) in the following nuclear fractions of a biological sample (e.g., mesenchymal stem cells) after being incubated at 37° C. for 24 hours in an incubation buffer having 100 mM trehalose, with 2% by weight DMSO being included in the incubation buffer during the last 2 hours of incubation: unbroken cells(N), mitochondrial fraction (M), and a lysosomal fraction (L). Graphs 390, 394 and 398 in FIG. 31, respectively, represent the distribution of solute (i.e., trehalose) in the following nuclear fractions of a biological sample (e.g., mesenchymal stem cells) after being incubated at 37° C. for 24 hours in an incubation buffer having 100 mM trehalose, with 2% by weight DMSO being included in the incubation buffer during the last 4 hours of incubation: unbroken cells(N), mitochondrial fraction (M), and a lysosomal fraction (L). Thus, including or adding DMSO in the incubation buffer for loading trehalose increases the uptake of trehalose from the incubation buffer, and improves the intracellular distribution of trehalose within the biological sample. Preferably, and as previously indicated, the incubation time for a biological sample in a loading solution comprising trehalose and DMSO is greater than about 8 hours, such as from about 8 hours to about 24 hours.

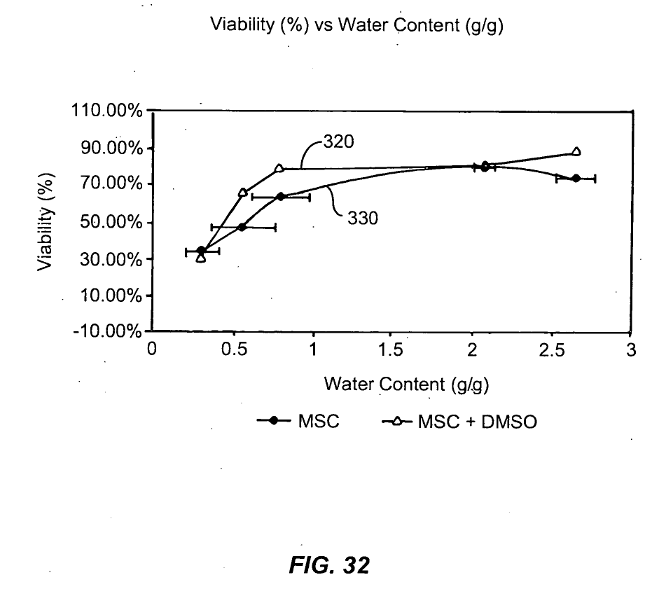

In other embodiments of the present invention, it has been discovered, as previously indicated, that DMSO aids in the recovery of biological samples following drying (e.g., air drying, vacuum drying, etc.) and rehydration, as broadly illustrated in FIG. 32 which is a graph of survival (% viability) vs. water content (gm. water/gm. dry weight) for a first batch of mesenchymal stem cells (MSC cells) loaded with trehalose and after air drying and rehydration, and for a second batch of mesenchymal stem cells (MSC cells) loaded with trehalose and DMSO and after air drying and rehydration. Example 14 below provides the more specific testing conditions and parameters which produced the graphical illustrations of FIG. 32. Broadly for the experiment in Example 14, DMSO was shown to aid the recovery of MSCs following air-drying and rehydration. All the MSCs were loaded with 100 mM trehalose for 24 hours. The experimental samples were also treated with 2% DMSO for the last three hours of the incubation. The dried samples were rehydrated with excess medium, and viability was assessed by trypan blue exclusion.

Graph 320 in FIG. 32 represents the % survival of the first batch of mesenchymal stem cells (MSC cells) after being incubated at 37° C. for 24 hours in an incubation buffer having 100 mM trehalose, with 2% by weight DMSO being included in the incubation buffer during the last 4 hours of incubation. Graph 330 in FIG. 32 represents the % survival of the second batch of mesenchymal stem cells (MSC cells) after being incubated at 37° C. for 24 hours in an incubation buffer having 100 mM trehalose and no DMSO and after air drying and rehydration. FIG. 32 broadly illustrates that cell survival increases (e.g., increases by from about 10% to about 40%) by adding DMSO to the drying buffer. FIG. 32 more specifically illustrates that after mesenchymal stem cells were loaded with trehalose (e.g., 25 mM to 800 mM trehalose) and DMSO (e.g., from about 0.10% by weight to about 25.0% by weight DMSO)while incubating at a temperature above about 250° C. (e.g., from about 35° C. to about 40° C.), and then dried (e.g., air dried) until the mesenchymal stem cells comprised a residual water content of greater than about 0.30 grams of water per gram of dry weight of mesenchymal stem cells (e.g., from about 0.30 grams of water per gram of dry weight of mesenchymal stem cells to about 2.2 grams of water per gram of dry weight of mesenchymal stem cells), survival (% viability) increases. Thus, adding DMSO to the drying buffer is a preferred drying technique for trehalose-loaded, mesenchymal stem cells if the residual water content of the trehalose-loaded, mesenchymal stem cells is maintained at greater than (or equal to) about 0.30 grams of water per gram of dry weight of mesenchymal stem cells (e.g., from about 0.30 grams of water per gram of dry weight of mesenchymal stem cells to about 3.0 grams water per gram of dry weight of mesenchymal stem cells). As shown in FIG. 32, the survival of the mesenchymal stem cells (i.e., the biological sample) is preferably at least about 50% (e.g., such as from about 50% to about 80%), more preferably at least about 80%.

As may be gathered from various aspects of the Figures, in preparing particularly preferred embodiments, platelets may be loaded with trehalose by-incubation at 37° C. for about four hours. The trehalose concentration in the loading buffer is preferably 35 mM, which results in an intracellular trehalose concentration of around 20 mM, but in any event is most preferably not greater than about 50 mM trehalose. At trehalose concentrations below about 50 mM, platelets have a normal morphological appearance.

Human platelets have a phase transition between 12° C. and 20° C. We found relatively poor loading when the platelets were chilled through the phase transition. Thus, in practicing the method described by U.S. Pat. No. 5,827,741, of which some of us are coinventors, only a relatively modest amount of trehalose may be loaded into platelets.

In this application, we have further investigated the phase transition in platelets and have found a second phase transition between 30° C. and 37° C. We believe that the excellent loading we obtain at about 37° C. is in some way related to this second phase transition. It may be that other oligosaccharides (other than trehalose) when loaded in this second phase transition in amounts analogous to trehalose could have similar effects.

In any case, it is fortuitous that the loading can be done at elevated temperatures in view of the fact that chilling platelets slowly—a requirement for using the first, or lower, phase transition between 20° C. and 12° C. to introduce trehalose —is well known to activate them (Tablin et al., J. Cell. Physiol., 168, 305313, 1996). Our relatively high temperature loading, regardless of the mechanism, is thus unexpectedly advantageous both by providing increased loading as well as surprisingly, obviating the activation problem.

Turning to FIG. 6, one sees that we have loaded other, larger molecules into the platelets. In FIG. 6 an illustrative large molecule (FITC dextran) was loaded into the platelets. This illustrates that a wide variety of water-soluble, therapeutic agents can be loaded into the platelets by utilizing the second phase transition, as we have shown may be done with trehalose and with FITC dextran, while still maintaining characteristic platelet surface receptors and avoiding platelet activation.

We have achieved loading efficiencies by practicing the invention with values as high as 61% after four hours incubation. The plateau is not yet reached after four hours. The high loading efficiency of trehalose is a strong indication that the trehalose is homogeneously distributed, and we expect similar results for loading other therapeutic agents. A loading efficiency of 61% in an external concentration of 25 mM corresponds to a cytosolic concentration of 15 mM.

We have found that the endocytotic uptake route is blocked at sugar concentrations above 0.1 M. Consequently, we prefer not to use sugar concentrations higher than about 50 mM in the loading buffer, because at some point above this value we have found swelling and morphological changes of the platelets. Thus, we have found that platelets become swollen after four hours incubation at 37° C. in 75 mM trehalose. Further, at concentrations higher than 50 mM the internal trehalose concentration begins to decrease. By contrast to embodiments of the present invention, the platelet method taught by Spargo et al., U.S. Pat. No. 5,736,313, loads with carbohydrate in the range beginning at about 100 mM and going up to 1.5 M. As noted, we find a high concentration of loading buffer, at least with trehalose, to lead to swelling and morphological changes.

The effective loading of platelets with trehalose is preferably conducted by incubating for at least about two hours, preferably for at least about four hours. After this loading, then the platelets are cooled to below their freezing point and lyophilized.

Before freezing, the platelets should be placed into a resting state. If not in the resting state, platelets would likely activate. In order to place the platelets in a resting state, a variety of suitable agents, such as calcium channel blockers, may be used. For example, solutions of adenine, adenosine or iloprost are suitable for this purpose. Another suitable agent is PGE1 (prostaglandin E1). It is important that the platelets are not swollen and are completely in the resting state prior to drying. The more they are activated, the more they will be damaged during freeze-drying.

After the platelets have been effectively loaded with trehalose and are in a resting state, then the loading buffer is removed and the platelets are contacted with a drying buffer.

The drying buffer should include trehalose, preferably in amounts up to about 100 mM. The trehalose in the drying buffer assists in spatially separating the platelet as well as stabilizing the platelet membranes on the exterior. The drying buffer preferably also includes a bulking agent (to further separate the platelets). Albumin may serve as a bulking agent, but other polymers may be used with the same effect. If albumin is used, it is preferably from the same species as the platelets. Suitable other polymers, for example, are water-soluble polymers such as HES (hydroxy ethyl starch) and dextran.

The trehalose loaded platelets in drying buffer are then cooled to a temperature below about −32° C. A cooling, that is, freezing, rate is preferably between −30° C. and −1° C./min. and more preferably between about −2° C./min to −5° C./min.

The lyophilization step is preferably conducted at a temperature below about −32° C., for example conducted at about −40° C., and drying may be continued until about 95 weight percent of water has been removed from the platelets. During the initial stages of lyophilization, the pressure is preferably at about 10×10⁻⁶ torr. As the samples dry, the temperature can be raised to be warmer than −32° C. Based upon the bulk of the sample, the temperature and the pressure it can be empirically determined what the most efficient temperature values should be in order to maximize the evaporative water loss. Freeze-dried compositions of the invention preferably have less than about 5 weight percent water.

The freeze-dried platelets may be used by themselves, dissolved in a physiologically acceptable solution, or may be a component of a biologically compatible (biocompatible) structure or matrix, which provides a surface on or by which the freeze-dried platelets are carried. The freeze-dried platelets can be, for example, applied as a coating to or impregnated in a wide variety of known and useful materials suitable as biocompatible structures for therapeutic 30 applications. The earlier mentioned U.S. Pat. No. 5,902,608, for example, discusses a number of materials useful for surgical aid, wound dressings, bandages, sutures, prosthetic devices, and the like. Sutures, for example, can be monofilament or braided, can be biodegradable or nonbiodegradable, and can be made of materials such as nylon, silk, polyester, cotton, catgut, homopolymers, and copolymers of glycolide and lactide, etc. Polymeric materials can also be cast as a thin film, sterilized, and packaged for use as a wound dressing. Bandages may be made of any suitable substrate material, such as woven or nonwoven cotton or other fabric suitable for application to or over a wound, may optionally include a backing material, and may optionally include one or more adhesive regions on the face surface thereof for securing the bandage over the wound.

The freeze-dried platelets, whether by themselves, as a component of a vial-compatible structure or matrix, and optionally including other dry or freeze-dried components, maybe packaged so as to prevent rehydration until desired. The packaging may be any of the various suitable packagings for therapeutic purposes, such as made from foil, metallized plastic materials, and moisture barrier plastics (e.g. high-density polyethylene or plastic films that have been created with materials such as SiOx), cooling the trehalose loaded platelets to below their freezing point, and lyophilizing the cooled platelets. The trehalose loading includes incubating the platelets at a temperature from greater than about 25° C. to less than about 40° C. with a trehalose solution having up to about 50 mM trehalose therein. The process of using such a dehydrated composition comprises rehydrating the platelets. The rehydration preferably includes a prehydration step, sufficient to bring the water content of the freeze-dried platelets to between about 20 weight percent and about 50 percent, preferably from about 20 weight percent to about 40 weight percent.

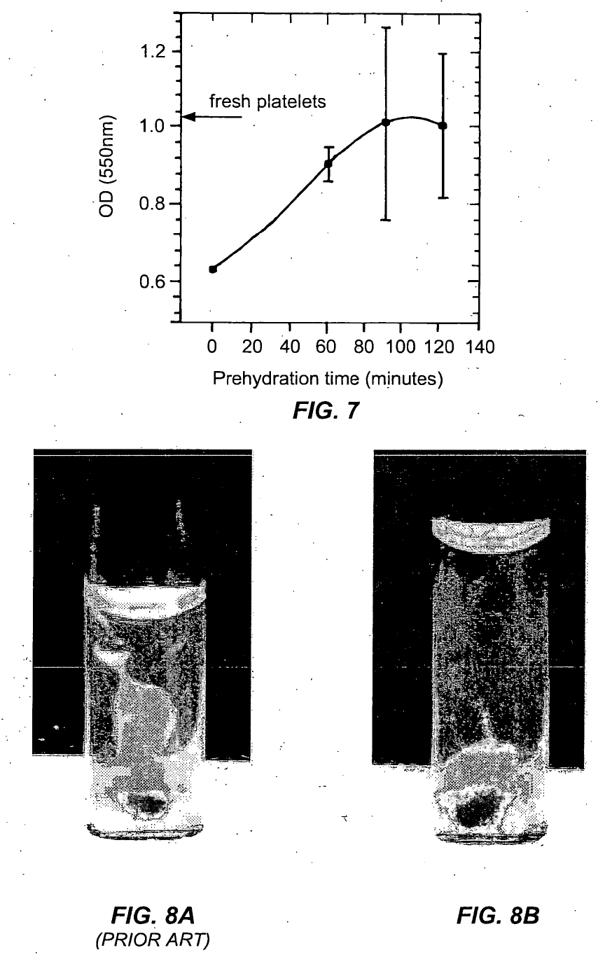

When reconstitution is desired, prehydration of the freeze-dried platelets in moisture saturated air followed by rehydration is preferred. Use of prehydration yields cells with a much more dense appearance and with no balloon cells being present. Prehydrated, previously lyophilized platelets of the invention resemble fresh platelets. This is illustrated, for example, by FIG. 7. As can be seen, the previously freeze-dried platelets can be restored to a condition that looks like fresh platelets.

Before the prehydration step, it is desirable to have diluted the platelets in the drying buffer to prevent aggregation during the prehydration and rehydration. At concentrations below about 3×10⁸ cells/ml, the ultimate recovery is about 70% with no visible aggregates. Prehydration is preferably conducted in moisture saturated air, most preferably is conducted at about 37° C. for about one hour to about three hours. The preferred prehydration step brings the water content of the freeze-dried platelets to between about 20 weight percent to about 50 weight percent.

The prehydrated platelets may then be fully rehydrated. Rehydration may be with any aqueous based solutions, depending upon the intended application. In one preferred rehydration, we used plasma, which resulted in about 90% recovery.

Since it is frequently desirable to dilute the platelets to prevent aggregation when the freeze-dried platelets are once again hydrated, it may then be desired or necessary for particular clinical applications to concentrate the platelets. Concentration can be by any conventional means, such as by centrifugation. In general, a rehydrated platelet composition will preferably have 10⁶ to 10¹¹ platelets per ml, more preferably 10⁸ to 10¹⁰ platelets per ml.

By contrast with the previous attempts at freeze drying platelets, we show here that with a very simple loading, freeze-drying and rehydration protocol one obtains platelets that are morphologically intact after rehydration, and have an identical response to thrombin as do fresh platelets. Moreover, the concentration of thrombin to give this response is a physiological concentration—1 U/ml.

For example, FIG. 8, panel (A), illustrates the clot formation for fresh platelets and in panel (B) for platelets that have been preserved and then rehydrated in accordance with this invention. The cell counts that were determined after three minutes exposure to thrombin were zero for both the fresh platelets and the previously freeze-dried platelets of the invention.

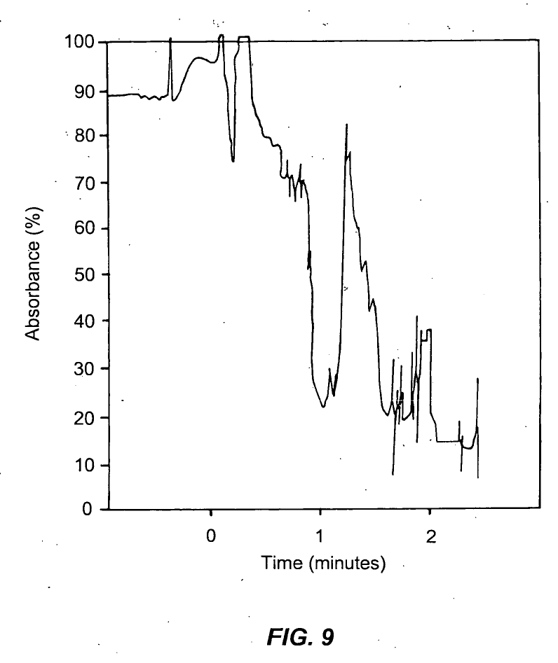

FIG. 9 graphically illustrates clotting as measured with an aggregometer. With this instrument one can measure the change in transmittance when a clot is formed. The initial platelet concentration was 250×10⁶ platelets/ml, and then thrombin (1 U/ml) was added and the clot formation was monitored with the aggregometer. The absorbance fell sharply and the cell count dropped, to below 2×10⁶ platelets/ml after three minutes, which was comparable to the results when the test was run with fresh platelets as a control.

Although platelets for use in embodiments of this invention preferably have had other blood components removed before freeze-drying, compositions and apparatuses of embodiments of the invention may also include a variety of additional therapeutic agents. For example, particularly for embodiments contemplated in hemostasis applications, antifungal and antibacterial agents are usefully included with the platelets, such as being admixed with the platelets. Embodiments can also include admixtures or compositions including freeze-dried collagen, which provides a thrombogenic surface for the platelets. Other components that can provide a freeze-dried extra-cellular matrix can be used, for example, components composed of proteoglycan. Yet other therapeutic agents that may be included in inventive embodiments are growth factors. When the embodiments include such other components, or admixtures, they are preferably in dry form, and most preferably are also freeze-dried. We also contemplate therapeutic uses of the composition where additional therapeutic agents may be incorporated into or admixed with the platelets in hydrated form. The platelets, as earlier mentioned, can also be prepared as to encapsulate drugs in drug delivery applications. If trehalose is also loaded into the platelet interiors, then such drug encapsulated platelets may be freeze-dried as has been earlier described.

The platelets should be selected of the mammalian species for which treatment is intended (e.g. human, equine, canine, feline, or endangered species), most preferably human. The injuries to be treated by applying hemostasis aids with the platelets include abrasions, incisions, burns, and may be wounds occurring during surgery of organs or of skin tissue. The platelets of the invention may be applied or delivered to the location of such injury or wound by any suitable means. For example, application of inventive embodiments to burns can encourage the development of scabs, the formation of chemotactic gradients, of matrices for inducing blood vessel growth, and eventually for skin cells to move across and fill in the burn.

For transfusion therapy, inventive compositions may be reconstituted (rehydrated) as pharmaceutical formulations and administered to human patients by intravenous injection. Such pharmaceutical formulations may include any aqueous carrier suitable for rehydrating the platelets (e.g., sterile, physiological saline solution, including buffers and other therapeutically active agents that may be included in the reconstituted formulation). For drug delivery, the inventive compositions will typically be administered into the blood stream, such as by i.v.

Embodiments of the present invention will be illustrated by the following set forth examples which are being given by way of illustration only and not by way of any limitation. All parameters such as concentrations, mixing proportions, temperatures, rates, compounds, etc., submitted in these examples are not to be construed to unduly limit the scope of the invention. Abbreviations used in the examples, and elsewhere, are as follows:

DMSO=dimethylsulfoxide

ADP=adenosine diphosphate

PGE1=prostaglandin E1

HES=hydroxy ethyl starch

FTIR=Fourier transform infrared spectroscopy

EGTA=ethylene glycol-bis(2-aminoethyl ether) N,N,N′,N′, tetra-acetic acid

TES=N-tris (hydroxymethyl) methyl-2-aminoethane-sulfonic acid

HEPES=N-(2-hydroxyl ethyl) piperarine-N′-(2-ethanesulfonic acid)

PBS=phosphate buffered saline

HSA=human serum albumin

BSA=bovine serum albumin

ACD=citric acid, citrate, and dextrose

MPCD=methyl-β-cyclodextrin

### Experimental

### EXAMPLE 1

Washing of Platelets. Platelet concentrations were obtained from the Sacramento blood center or from volunteers in our laboratory. Platelet rich plasma was centrifuged for 8 minutes at 320×g to remove erythrocytes and leukocytes. The supernatant was pelleted and washed two times (480×g for 22 minutes, 480×g for 15 minutes) in buffer A (100 MM NaCl, 10 MM KCl, 10 mM EGTA, 10 mM imidazole, pH 6.8). Platelet counts were obtained on a Coulter counter T890 (Coulter, Inc., Miami, Fla.).

Loading of Lucifer Yellow CH into Platelets. A fluorescent dye, lucifer yellow CH (LYCH), was used as a marker for penetration of the membrane by a solute. Washed platelets in a concentration of 1-2×10⁹ platelets/ml were incubated at various temperatures in the presence of 1-20 mg/ml LYCH. Incubation temperatures and incubation times were chosen as indicated. After incubation the platelets suspensions were spun down for 20× at 14,000 RPM (table centrifuge), resuspended in buffer A, spun down for 20 s in buffer A and resuspended. Platelet counts were obtained on a Coulter counter and the samples were pelleted (centrifugation for 45 s 25 at 14,000 RPM, table centrifuge). The pellet was lysed in 0.1% Triton buffer (10 mM TES, 50 mM KCl, pH 6.8). The fluorescence of the lysate was measured on a Perkin-Elmer LSS spectrofluorimeter with excitation at 428 nm (SW 10 nm) and emission at 530 run (SW 10 nm). Uptake was calculated for each sample as nanograms of LYCH per cell using a standard curve of LYCH in lysate buffer. Standard curves of LYCH, were found to be linear up to 2000 run ml⁻¹.

Visualization of cell-associated Lucifer Yellow. LYCH loaded platelets were viewed on a fluorescence microscope (Zeiss) employing a fluorescein filter set for fluorescence microscopy. Platelets were studied either directly after incubation or after fixation with 1% paraformaldehyde in buffer. Fixed cells were settled on poly-L-lysine coated cover slides and mounted in glycerol.

Loading of Platelets with Trehalose. Washed platelets in a concentration of 1-2 10⁹ platelets/ml were incubated at various temperatures in the presence of 1-20 mg/ml trehalose. Incubation temperatures were chosen from 4° C. to 37° C. Incubation times were varied from 0.5 to 4 hours. After incubation the platelet solutions were washed in buffer A two times (by centrifugation at 14,000 RPM for 20 s in a table centrifuge). Platelet counts were obtained on a coulter counter. Platelets were pelleted (45 S at 14,000 RPM) and sugars were extracted-from the pellet using 80% methanol. The samples were heated for 30 minutes at 80° C. The methanol was 10 evaporated with nitrogen, and the samples were kept dry and redissolved in H₂O prior to analysis. The amount of trehalose in the platelets was quantified using the anthrone reaction (Umbreit et al., Mamometric and Biochemical Techniques, 5th Edition, 1972). Samples were redissolved in 3 ml H₂O and 6 ml anthrone reagents (2 g anthrone dissolved in 10M sulfuric acid). After vortex mixing, the samples were placed in a boiling water bath for 3 minutes. Then the samples were cooled on ice and the absorbance was measured at 620 nm on a Perkin Elmer spectrophotometer. The amount of platelet associated trehalose was determined using a standard curve of trehalose. Standard curves of trehalose were found to be linear from 6 to 300 μg trehalose per test tube.

Quantification of Trehalose and LYCH Concentration. Uptake was calculated for each sample as micrograms of trehalose or LYCH per platelet. The internal trehalose concentration was calculated assuming a platelet radius of 1.2 μm and by assuming that 50% of the platelet volume is taken up by the cytosol (rest is membranes). The loading efficiency was determined from the cytosolic trehalose or LYCH concentration and the concentration in the loading buffer.

FIG. 1 shows the effect of temperature on the loading efficiency of trehalose into human platelets after a 4 hour incubation period with 50 mM external trehalose. The effect of the temperature on the trehalose uptake showed a similar trend as the LYCH uptake. The trehalose uptake is relatively low at temperatures of 22° C. and below (below 5%), but at 37° C. the loading efficiency of trehalose is 35% after 4 hours.

When the time course of trehalose uptake is studied at 37° C., a biphasic curve can be seen (FIG. 2). The trehalose uptake is initially slow (2.8×10⁻¹¹ mol/m²s from 0 to 2 hours), but after 2 hours a rapid linear uptake of 3.3×10⁻¹⁰ mol/m²s can be observed. The loading efficiency increases up to 61% after an incubation period of 4 hours. This high loading efficiency is a strong indication that the trehalose is homogeneously distributed in the platelets rather than located in pinocytosed vesicles.

The uptake of trehalose as a function of the external trehalose concentration is shown in FIG. 3. The uptake of trehalose is linear in the range from 0 to 30 mM external trehalose. The highest internal trehalose concentration is obtained with 50 mM external trehalose. At higher concentrations than 50 mM the internal trehalose concentration decreases again. Even when the loading buffer at these high trehalose concentrations is corrected for isotonicity by adjusting the salt concentration, the loading efficiency remains low. Platelets become swollen after 4 hours incubation in 75 mM trehalose.

The stability of the platelets during a 4 hours incubation period was studied using microscopy and flow cytometric analysis. No morphological changes were observed after 4 hours incubation of platelets at 37° C. in the presence of 25 mM external trehalose. Flow-cytometric analysis of the platelets showed that the platelet population is very stable during 4 hours incubation. No signs of microvesicle formation could be observed after 4 hours incubation, as can be judged by the stable relative proportion of microvesicle gated cells (less than 3%). The formation of microvesicles is usually considered as the first sign of platelet activation (Owners et al., Trans. Med. Rev., 8, 27-44, 1994). Characteristic antigens of platelet activation include: glycoprotein 53 (gp53, a lysosomal membrane marker), PECAM-1 (platelet endothelial cell adhesion molecule-1, an alpha granule constituent), and P-selection (an alpha granule membrane protein).

### EXAMPLE 2

Washing Platelets. Platelets were obtained from volunteers in our laboratory. Platelet rich plasma was centrifuged for 8 minutes at 320×g to remove erythrocytes and leukocytes. The supernatant was pelleted and washed two times (480×g for 22 minutes, 480×g for 15 minutes) in buffer A (100 mM NaCl, 10 mM KCl, 10 mM EGTA, 10 mM imidazole, 10 ug/ml PGE1, pH 6.8). Platelet counts were obtained on a Coulter counter T890 (Coulter, Inc., Miami, Fla.).

Loading Platelets with Trehalose. Platelets were loaded with trehalose as described in Example 1. Washed platelets in a concentration of 1-2×10⁹ platelets/ml were incubated at 37° C. in buffer A with 35 mM trehalose added. Incubation times were typically 4 hours. The samples were gently stirred for 1 minute every hour. After incubation the platelet solutions were pelleted (25 sec in a microfuge) and resuspended in drying buffer (9.5 mM HEPES, 142.5 mM NaCl, 4.8 mM KC1, 1 MM MgCl₂, 30 mM Trehalose, 1% Human Serum Albumin, 10 ug/ml PGE1). In the aggregation studies no PGE1 was added in the drying buffer. Trehalose was obtained from Pfahnstiehl. Human serum albumin was obtained from Sigma.

Freezing and Drying. Typically 0.5 ml platelet suspensions were transferred in 2 ml Nunc cryogenic vials and frozen in a Cryomed controlled freezing device. Vials were frozen from 22° C. to −40° C. with freezing rates between −30 and −1° C./min and more often between −5 and −2° C./min. The frozen solutions were transferred to a −80° C. freezer and kept there for at least half an hour. Subsequently the frozen platelet suspensions were transferred in vacuum flasks that were attached to a Virtis lyophilizes. Immediately after the flasks were hooked up to the lyophilizer, they were placed in liquid nitrogen to keep the samples frozen until the vacuum returned to 20×10⁻⁶ Torr, after which the samples were allowed to warm to the sublimation temperature. The condenser temperature was −45° C. Under these conditions, sample temperature during primary drying is about −40° C., as measured with a thermocouple in the sample. It is important to maintain the sample below Tg for the excipient during primary drying (−32° C. for trehalose).

Rehydration. Vials with originally 0.5 ml platelet suspension were rehydrated in 1 ml PBS buffer/water (1/1). PBS buffer was composed of 9.4 mM Na₂HP0₄, 0.6 mM KH₂PO₄, 100 mM NaCl, pH7.2). In a few experiments PGE1 was added to the rehydration buffer in a condition of 10 μg/ml or rehydration was performed in plasma/water (1/1).

Prehydration. Platelet lyophilisates were prehydrated in a closed box with moisture saturated air at 37° C. Prehydration times were between 0 and 3 hours.

Recovery. The numerical recovery of lypophilized and (p)rehydrated platelets was determined by comparing the cell count with a Coulter count T890 (Coulter, Inc., Miami, Fla.) before drying and after rehydration. The morphology of the rehydrated platelets was studied using a light microscope. For this purpose platelets were fixed in 2% paraformaldehyde or gutaraldehyde and allowed to settle on poly-L-lysine coated coverslides for at least 45 minutes. After this the coverslides were mounted and inspected under the microscope. The optical density of freeze-dried and rehydrated platelets was determined by measuring the absorbance of a platelet suspension of 1.0×10⁸ cells/ml at 550 nm on a spectrophotometer.

Aggregation studies. Dried platelets were rehydrated (after 2 hour prehydration) with 2 aliquots of platelet free plasma (plasma was centrifuged for 5 minutes at 3800×g) diluted with water in 1/1 ratio. Half ml aliquots of this platelet suspension were transferred to aggregation cuvettes with a magnetic stirrer. The response of the platelets to thrombin was tested by adding thrombin (1 U/ml) to the platelet suspension at 37° C. under stirring conditions. After 3 minutes thrombin treated platelet suspensions were inspected for clots and cell counts were done on a Coulter Counter T890.

Direct rehydration tends toward cell lysis and prehydration leads to aggregation when the cell concentration is 10⁹ cells/ml in the drying buffer. We found also that recovery of prehydrated and rehydrated platelets depends on the cell concentration in the drying buffer. The recovery drops to very low values if the cell concentration is higher than 3×10⁸ cells/ml. At concentrations below 3×10⁸ cells/ml, the recovery is around 70%, and no aggregates were visible. Prehydration resulted in denser cells and the absence of balloon cells.

Longer prehydration times than 90 minutes did not further improve the cellular density, but slightly activated the platelets. The water content of the pellet increases with increasing prehydration time, and preferably is between about 35% and 50% at the moment of rehydration.

At higher water contents than 50% water droplets become visible in the lyophilisate (which means that the platelets are in a very hypertonic solution).

As described by Example 1, platelets were loaded with trehalose by incubation at 37° C. for 4 hours in buffer A with 35 mM trehalose, which yielded platelets with intracellular trehalose concentration of 15-25 mM. After incubation, the platelets were transferred to drying buffer with 30 mM trehalose and 1% HSA as the main excipients.

The directly rehydrated platelets had a high numerical recovery of 85%, but a considerable fraction (25-50%) of the cells was partly lysed and had the shape of a balloon. Directly rehydrated platelets were overall less dense when compared with fresh platelets.

The numerical recovery of platelets that were prehydrated in moisture saturated air was only 25% when the platelet concentration was 1×10⁹ cells/ml in the drying buffer. This low recovery was due to aggregates that were formed during the prehydration period. But the cells that were not aggregated were more dense than the directly rehydrated platelets and resembled that of fresh platelets.

Since it appears desirable to dilute the platelets to prevent aggregation during the prehydration step, it may be necessary for clinical applications to concentrate the platelets following rehydration. We therefore also tested the stability of the rehydrated platelets with respect to centrifugation and found that the directly rehydrated platelets had 50% recovery after centrifugation, while the prehydrated ones had 75% recovery following centrifugation. Thus, we conclude that the inventive platelets can be concentrated without ill effect.

### EXAMPLE 3

We view trehalose as the main lyoprotectant in the drying buffer. However, other components in the drying buffer, such as albumin, can improve the recovery. In the absence of external trehalose in drying buffer, the numerical recovery is only 35%. With 30 mM trehalose in the drying buffer the recovery is around 65%. A combination of 30 mM trehalose and 1% albumin gave a numerical recovery of 85%.

### EXAMPLE 4

Typically 0.5 ml platelet suspensions were transferred in 2 ml Nunc cryogenic vials and frozen in a Cryomed controlled freezing device. Vials were frozen from 22° C. to −40° C. with freezing rates between −30° C./min and −1° C./min and more often between −5° C. and −2° C./min. The frozen solutions were transferred to a −80° C. freezer and kept there for at least half an hour. Subsequently the frozen platelet suspensions were transferred in vacuum flasks that were attached to a Virtus lyophilizer. Immediately after the flasks were hooked up to the lyophilizer, they were placed in liquid nitrogen to keep the samples frozen until the vacuum returned to 20×10⁻⁶ Torr, after which the samples were allowed to warm to the sublimation temperature. The condenser temperature was −45° C. Under these conditions, sample temperature during primary drying is about −40° C., as measured with a thermocouple in the sample. It is important to maintain the sample below Tg. for the excipient during primary drying (−32° C. for trehalose). Only minor differences in recovery were found as a function of the freezing rate. The optimal freezing rate was found to be between 2° C. and 5° C./minute.

### EXAMPLE 5

Response of freeze-dried platelets to thrombin (1 U/ml) was compared with that of fresh platelets. The platelet concentration was 0.5×10⁸ cells/ml in both samples. 500 μl platelets solution was transferred into aggregation vials. Thrombin was added to the samples and the samples were stirred for 3 minutes at 37° C. The cell counts that were determined after 3 minutes were 0 for both the fresh and the freeze-dried platelets. The response to thrombin was determined by a cleavage in glycoprotein lb-(GPlb). This was detected by using monoclonal antibodies and flow cytometry. Thus, the pattern seen after addition of thrombin was a reduced amount of GP lb on the platelet surface.

The response of lyophilized, prehydrated, and rehydrated platelets (Examples 1 and 2) to thrombin (1 U/ml) was found to be identical compared with that of fresh platelets. In both fresh and rehydrated platelets a clot was formed within 3 minutes at 37° C. These clots are illustrated by FIG. 8, panels (A) and (B). When cell counts were done with the Coulter counter, we found no cells present, indicating that all platelets participated in forming the clot illustrated in panel (B).

### EXAMPLE 6

Reactions with other agonists were studied. Platelet suspensions of the inventive platelets were prepared with 50×10⁶ platelets/ml. Different agonists were then added and subsequently counted with a Coulter counter to determine the percentage of platelets involved in the visually observable clot formation. The cell count was between 0 and 2×10⁶ platelets/ml: after 5 minutes with 20 μg/ml collagen; after 5 minutes with 20 μM ADP; after 5 minutes with 1.5 mg/ml ristocetin. This means that the percentage of platelets that are involved in clot formation is between 95-100% for all the agonists tested. The agonist concentrations that were used are all physiological. In all cases the percentage of clotted platelets was the same as fresh control platelets.

### EXAMPLE 7

Trehalose and sucrose solutions were prepared in water (100 mM). The solutions were heated to 70° C. for 30 minutes, after which the solutions were analyzed by HPLC (high performance liquid chromatography. Trehalose survived this treatement down to pH 1.0, while most of the sucrose was hydrolyzed to glucose and fructose at pH as high as 5. At lower temperatures this pattern persisted, although the time required to hydrolyze the sucrose increased. It is well established that the pH in lysosomes is 4-5, so it follows that sucrose if likely to be degraded in lysosomes, while trehalose should escape damage. The residence time in the lysosomes would be expected to be critical in this regard. At 370 C., for example, sucrose would experience minimal degradation if the residence time is 10 minutes, but degradation would be extensive if the residence time were on the order of hours.

### EXAMPLE 8

Membranes become leaky at the pH found in lysosomes. Liposomes composed of the phospholipids POPC (palmitoyloleyoylphosphatidylcholine) and PS (phosphatidylserine) (9:1) were prepared by extrusion through 100 nm filters. A marker for permeability, the fluorescent marker carboxyfluorescein (CF) was trapped in liposomes at a concentration of 0.5 M during the extrusion. External CF was removed by passing the liposomes through a Sephadex column. The liposomes were then subjected to decreased pH. CF is fluorescent, but self-quenching at the concentration at which it was trapped in the lipsosomes. When the trapped CF leaks into the external medium, it becomes diluted, and fluorescence increases. From the rate of increase in fluorescence it is possible to deduce the permeability.

### EXAMPLE 9

Leakage from lysosomes in vivo is in reasonable agreement with the in vitro data. Cells were incubated in a fluorescent probe, Lucifer yellow. This particular probe was chosen as a tracer since it is approximately the same size as a disaccharide. The cells were washed free of extracellular Lucifer yellow and then observed by fluorescence microscopy. The results are shown in FIGS. 23-26. When the cells were incubated in the dye for 1 to 3.5 hours, punctuate staining was clearly seen, indicating the presence of the dye in endosomes or lysosomes. However, by 5 hours much of the punctuate staining disappeared and the cytoplasm acquired a uniform fluorescence. Thus, 3.5 to 5 h hours are required for appreciable leakage to occur. Thus, there is reasonable agreement between the two measurements.

### EXAMPLE 10

Trehalose survives passage through lysosomes in vivo, while other sugars do not. Platelet cells were incubated for four hours in 100 mM trehalose, sucrose, or raffinose, respectively. The platelet cells were then homogenized in 60% methanol, from which the large particles were pelleted by centrifugation. The supernatant was removed, and analyzed by HPLC. The results showed that trehalose was recovered intact, with no evidence of degradation. Raffinose appeared to be completely hydrolyzed. Sucrose was partially hydrolyzed, but significant amounts of intact sucrose were obtained, nevertheless. It may well be that the difference between raffinose and sucrose lies in the fact that raffinose is a trisaccharide and thus might be expected to leak across the lysosomal membrane more slowly than does sucrose. Thus, with increased residence time hydrolysis would go further towards completion. Even a small amount of hydrolysis might not be acceptable; the monosaccharides that are produced as a result of the hydrolysis are all reducing sugars, and all show the Maillard reaction with dry proteins, a reaction that denatures the protein irreversibly.

### EXAMPLE 11

Human mesenchymal stem cells were grown to approximately 90% confluence in T-75 flasks. They were loaded with trehalose by incubating the attached cells in DMEM with the addition of 100 mM trehalose for 24 h at 37° C., a procedure which leads to an internal trehalose concentration in the range of 15-25 mM. The cells were harvested by trypsinization according to standard protocols. Briefly, the medium was removed from the cultures and they were washed one time with 5 mL DPBS. Trypsin (3 mL of 0.05% in 0.53 mM EDTA-4Na) was added to the culture for ˜4 min and the flasks were rapped to dislodge the cells. Medium (7 mL) was added to stop the reaction, and the cells were pelleted by centrifugation at 176×g for 5 min. The pellet was suspended in 10 mL DPBS and the centrifugation step was repeated. For freeze-drying, the cells were transferred into freeze-drying buffer (130 mM NaCl, 10 mM HEPES (pH 7.2), 5 mM KCl, 150 mM trehalose, and 5.7% BSA (w/v)). Samples (50 uL) were aliquotted into 1.5 mL Eppendorf microfuge tubes and frozen in a −80° C. freezer. The samples were lyophilized using a Virtis 25SL Freezemobile. For air-drying, the samples were transferred into air-drying buffer (10 mM Hepes, 5 mM KCL, 65 mM NaCl, 150 mM Trehalose, and 5.7% BSA with pH 7.2). Samples (0.5 mL) were aliquotted into 35 mm Petri dishes and air-dried uncovered in a ThermoForma biosafety cabinet in specific marked locations in the center of the hood over 0-24 hours. At various time points during drying, samples were removed for viability and water content analyses. Water contents were measured gravimetrically. For viability measurements, samples were rehydrated with 1 mL medium. 50 uL of cellular suspension was mixed with 50 uL trypan blue and incubated at room temperature for 3 min. Cells were visualized at 10× by light microscopy on a hemacytometer and counted using five counts of 50-100 cells per 1 mm² hemocytometer grid square for each sample. Percent viability was calculated as the number of cells excluding the dye divided by the total number of cells. The graphs in FIG. 27 were produced by plotting viability as a function of water content.

### EXAMPLE 12

This Example 12 is to provide and present the more specific testing conditions and parameters which produced the graphical illustrations of FIGS. 28-30.

MSCs were incubated with 10 mM LYSH for 5 hours in the presence or absence of DMSO, washed and examined by fluorescence microscopy. In the control sample (FIG. 28) in which no DMSO was present, the LYCH fluorescence was seen predominately within endosomes, as indicated by the punctuate staining. When 2% DMSO was included for the last 30 minutes of the incubation, a slightly more diffuse staining was seen (FIG. 29). The most dramatic result, however, was seen when 2% DMSO was included with the LYCH for the entire 5 hour incubation (FIG. 30). In this case, although some punctuate staining was still visible, diffuse LYCH staining was seen throughout the cytoplasm. This result indicates that DMSO may provide some benefit to the cells by aiding in the release of solutes from the endosomes and allowing a more homogeneous intracellular distribution.

Thus, recapitulating, to visualize LYCH uptake and the effect of DMSO on LYCH distribution, cells were plated in 2-well LabTek CC2 glass slides, and grown for 5-7 days until they reached −60% confluence. They were then incubated in MSC medium with 10 mM LYCH for 5 hours at 37° C. DMSO (2%) was included in the incubation either for the last 30 min of the 5-h period, for the entire 5-h period or not at all (control). Following the incubation, cells were washed three times with 1.5 mL DPBS and were fixed in 1% paraformaldehyde in DPBS for one hour at 22° C. Cells were mounted with Aqua-Poly/Mount), and observed and photographed using an Olympus BX30 microscope equipped with a Zeiss Axiocam running Axio Vison 3.1 software.

### EXAMPLE 13

Example 13 is to present and to provide the more specific testing conditions and parameters which produced the graphical illustrations of FIG. 31.

In order to determine if DMSO can aid the intracellular distribution of trehalose after endocytotic uptake, we conducted loading experiments under several different DMSO treatment conditions. MSCs were grown and loaded in the attached state with medium containing 100 mM trehalose for 24 h. This leads to an intracellular trehalose concentration in the range of 15-25 mM. DMSO (2%) was included in the loading medium for the full 24 h, or only for the last 2 or 4 h. The control was treated with trehalose alone. The cells were disrupted using a Dounce homogenizer and fractionated by differential centrifugation. Briefly, centrifugation at 400×g for 12 min gives the nuclear pellet; centrifugation at 1500×g for 10 min gives the mitochondrial pellet; and centrifugation at 10,000×g for 20 min gives the lysosomal pellet. Each fraction was extracted with 80% methanol, by heating to 80° C. for 1 h and analyzed by HPLC. FIG. 31 shows the results as the percent of total trehalose for each sample found in each organellar fraction. It is important to note that the nuclear fraction also contains the residual undisrupted whole cells. These data indicate that, in the control samples, most of the trehalose was found in the nuclear/whole cell fraction with less in each of the mitochondrial and lysosomal fractions. However, in the sample treated for the full 24 h with 2% DMSO, the mitochondrial and lysosomal fractions show a sharp increase in trehalose compared to the nuclear/whole cell fraction. This suggests that DMSO does indeed aid in increasing the intracellular distribution of trehalose after endocytotic loading. The shorter treatments with DMSO, however, show much less effect. In both the 2 and 4 hour DMSO treatments, the trehalose found in the mitochondrial and lysosomal fractions is not different from the control.

### EXAMPLE 14

Example 14 is to present and to provide the more specific testing conditions and parameters which produced the graphical illustrations of FIG. 32.

Human mesenchymal stem cells were grown to approximately 90% confluence in T-75 flasks. They were loaded with trehalose by incubating the attached cells in DMEM with the addition of 100 mM trehalose for 24 h at 37° C., a procedure which leads to an internal trehalose concentration in the range of 15-25 mM. DMSO (2%) was included in the loading medium for the final 3 h of the incubation or not at all (control). The cells were harvested by trypsinization according to standard protocols. Briefly, the medium was removed from the cultures and they were washed one time with 5 mL DPBS. Trypsin (3 mL of 0.05% in 0.53 mM EDTA-4Na) was added to the culture for ˜4 min and the flasks were rapped to dislodge the cells. Medium (7 mL) was added to stop the reaction, and the cells were pelleted by centrifugation at 176×g for 5 min. The pellet was suspended in 10 mL DPBS and the centrifugation step was repeated. For air-drying, the samples were transferred into air-drying buffer (10 mM Hepes, 5 mM KCL, 65 mM NaCl, 150 mM Trehalose, and 5.7% BSA with pH 7.2). Samples (0.5 mL) were aliquotted into 35 mm Petri dishes and air-dried uncovered in a ThermoForma biosafety cabinet in specific marked locations in the center of the hood over 0-24 hours. At various time points during drying, samples were removed for viability and water content analyses. Water contents were measured gravimetrically. For viability measurements, samples were rehydrated with 1 mL medium. 50 uL of cellular suspension was mixed with 50 uL trypan blue and incubated at room temperature for 3 min. Cells were visualized at 10× by light microscopy on a hemacytometer and counted using five counts of 50-100 cells per 1 mm² hemocytometer grid square for each sample. Percent viability was calculated as the number of cells excluding the dye divided by the total number of cells. Viability was plotted as a function of water content.

### EXAMPLE 15

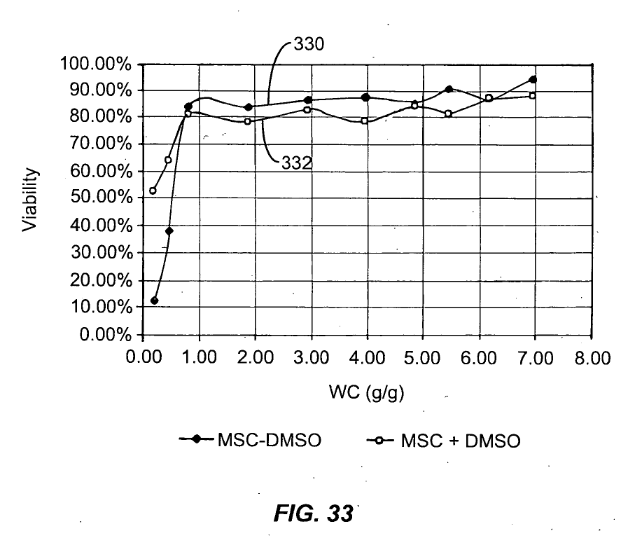

Example 15 is to present and to provide the specific testing-conditions and parameters which produced the graphical illustrations of FIG. 33, which reflect that DMSO improves viability following vacuum-drying. Graph 330 in FIG. 33 represents MSC with no DMSO. Graph 332 (MSC+DMSO) represents MSC cells which were treated with MSO at the end of the trehalose incubation.

Broadly, in this experiment DMSO is shown to aid the recovery of MSCs following vacuum-drying and rehydration. All the MSCs were loaded with 100 mM trehalose for 24 hours. The experimental samples were also treated with 2% DMSO for the last three hours of the incubation. The dried samples were rehydrated with excess medium, and viability was assessed by trypan blue exclusion.

More specifically in this experiment, human mesenchymal stem cells were grown to approximately 90% confluence in T-75 flasks. They were loaded with trehalose by incubating the attached cells in DMEM with the addition of 100 mM trehalose for 24 h at 37° C., a procedure which leads to an internal trehalose concentration in the range of 15-25 mM. DMSO (2%) was included in the loading medium for the final 3 h of the incubation or not at all (control). The cells were harvested by trypsinization according to standard protocols. Briefly, the medium was removed from the cultures and they were washed one time with 5 mL DPBS. Trypsin (3 mL of 0.05% in 0.53 mM EDTA-4Na) was added to the culture for ˜4 min and the flasks were rapped to dislodge the cells. Medium (7 mL) was added to stop the reaction, and the cells were pelleted by centrifugation at 176×g for 5 min. The pellet was suspended in 10 mL DPBS and the centrifugation step was repeated. For vacuum-drying, the samples were transferred into air-drying buffer (10 mM Hepes, 5 mM KCL, 65 mM NaCl, 150 mM Trehalose, and 5.7% BSA with pH 7.2). Samples (50 uL) were aliquotted into the caps of Eppendorf microfuge tubes and subjected to house vacuum (˜23 in Hg) for a period of 0-3 hours. At various time points during drying, samples were removed for viability and water content analyses. Water contents were measured gravimetrically. For viability measurements, samples were rehydrated to a total volume of 150 uL with medium. A small aliquot (150 uL) of cellular suspension was mixed with propidium iodide (to a final concentration of 2 ug/mL) and incubated at room temperature for at least 10 min. Cells were visualized at 10× by fluorescence microscopy on a hemacytometer and counted using at least four counts of 50-100 cells per 1 mm² hemocytometer grid square for each sample. Percent viability was calculated as the number of cells excluding the dye divided by the total number of cells, as counted using light microscopy. Viability was plotted as a function of water content for MSC with no DMSO and for MSC plus DMSO to produce the graphs 330 and 332 of FIG. 33.

### Conclusion

Embodiments of the present invention provide that trehalose, a sugar found at high concentrations in organisms that normally survive dehydration, can be used to preserve biological structures in the dry state. Human blood platelets can be loaded with trehalose under specified conditions, and the loaded cells can be freeze dried with excellent recovery. Additional embodiments of the present invention provide that trehalose may be used to preserve nucleated (eukaryotic) cells.

While the present invention has been described herein with reference to particular embodiments thereof, a latitude of modification, various changes and substitutions are intended in the foregoing disclosure, and it will be appreciated that in some instances some features of the invention will be employed without a corresponding use of other features without departing from the scope and spirit of the invention as set forth. Therefore, many modifications may be made to adapt a particular situation or material to the teachings of the invention without departing from the essential scope and spirit of the present invention. It is intended that the invention not be limited to the particular embodiment disclosed as the best mode contemplated for carrying out this invention, but that the invention will include all embodiments and equivalents falling within the scope of the appended claims.

What is claimed is:

1. A process for loading a biological sample comprising;
loading by fluid phase endocytosis a biological sample with a solute and dimethylsulfoxide to produce an internally loaded biological sample.

2. The process of claim 1 wherein said loading a biological sample by fluid phase endocytosis comprises fusing within the biological sample a first matter with a second matter to produce a fused matter.

3. The process of claim 2 wherein said first matter comprises the solute and dimethylsulfoxide.

4. The process of claim 2 wherein said first matter comprises a vesicle having the solute and dimethylsulfoxide.

5. The process of claim 2 wherein said second matter comprises a lysosome.

6. The process of claim 4 wherein said second matter comprises a lysosome.

7. The process of claim 2 wherein said fused matter comprises the solute and dimethylsulfoxide.

8. The process of claim 6 wherein said fused matter comprises the solute and dimethylsulfoxide.

9. The process of claim 2 wherein said loading a biological sample by fluid phase endocytosis additionally comprises transferring the solute and dimethylsulfoxide from the fused matter within the biological sample.

10. The process of claim 8 wherein said loading a biological sample by fluid phase endocytosis additionally comprises transferring the solute and dimethylsulfoxide from the fused matter within the biological sample.

11. The process of claim 9 wherein the solute and dimethylsulfoxide is transferred from the fused matter into a cytoplasm within the biological sample.

12. The process of claim 10 wherein the solute and dimethylsulfoxide is transferred from the fused matter into a cytoplasm within the biological sample.

13. The process of claim 2 wherein said fused matter comprises a lower pH than a pH of the first matter.

14. The process of claim 1 wherein said biological sample includes a biological sample selected from a group of biological samples comprising a platelet and a cell.

15. The process of claim 1 wherein said solute comprises trehalose.

16. A biological sample produced in accordance with the process of claim 1.

17. A process for preparing a dehydrated biological sample comprising:
providing a biological sample selected from a mammalian species;
loading the biological sample with a solute and dimethylsulfoxide to produce a loaded biological sample; and
drying the loaded biological sample to produce a dehydrated biological sample.

18. The process of claim 17 wherein said loading of the biological sample with a solute and dimethylsulfoxide comprises loading by fluid phase endocytosis of the biological sample with an oligosaccharide and dimethylsulfoxide from an oligosaccharide solution having the oligosaccharide and the dimethylsulfoxide.

19. The process of claim 18 wherein said oligosaccharide comprises trehalose.

20. The process of claim 19 wherein said drying of the loaded biological sample comprises drying the biological sample until the loaded biological sample has a water content ranging from about 0.3 grams of water per gram of dry weight biological sample to about 2.7 grams of water per gram of dry weight biological sample.

21. The process of claim 18 wherein said oligosaccharide solution comprises at least about 0.10 weight percent of dimethylsulfoxide.

22. The process of claim 20 wherein said drying comprises air drying.

23. The process of claim 17 wherein said biological sample comprises mesenchymal stem cells.

24. A method for increasing the survival of a biological sample comprising:
providing a biological sample;
loading the biological sample with a carbohydrate and dimethylsulfoxide to produce a loaded biological sample; and
drying the loaded biological sample while maintaining a residual water content in the biological sample of at least about 0.01 gram water per gram of dry weight of biological sample to increase survival of the biological sample.

25. The method of claim 24 additionally comprising storing the dehydrated loaded biological sample to produce a stored biological sample; and
rehydrating the stored biological sample.

26. The method of claim 24 wherein said biological sample comprises a mammalian biological sample.

27. The method of claim 24 wherein said drying comprises drying the biological sample until the loaded biological sample has a water content ranging from about 0.3 grams of water per gram of dry weight biological sample to about 2.7 grams of water per gram of dry weight biological sample.

28. A process for improving intracellular distribution of a solute in a biological sample comprising:
providing a biological sample; and
loading the biological sample with a carbohydrate and dimethylsulfoxide to produce a loaded biological sample having improved intracellular distribution over the biological sample having been loaded with the carbohydrate but without the dimethylsulfoxide.

29. The process of claim 28 wherein said biological sample comprises a fraction selected from the group of fractions comprising a mitochondrial fraction, a lysosomal frantion, and mixtures thereof.

30. The process of claim 29 wherein said intracellular distribution is improved in said fraction.

31. A method for increasing the survival of a biological sample comprising:
providing a biological sample;
loading the biological sample with a carbohydrate to produce a loaded biological sample; and
air drying the loaded biological sample while maintaining a residual water content in the biological sample of less than or equal to about 3.0 grams of water per gram of dry weight of biological sample to increase survival of the biological sample over the biological sample having been freeze-dried.

32. The method of claim 31 wherein said biological sample comprises a mesenchymal stem cell.

33. A solution for increasing the distribution of a solute in a biological sample comprising a solute, and at least about 0.10% by weight of dimethylsulfoxide.
